SEQUENCE LISTING (72 sequences)

SEQ ID NO: 1 - protein, 26 aa, Homo sapiens
AYFGASLCSVDVDSNGSTDLVLIGAP

SEQ ID NO: 2 - protein, 26 aa, Homo sapiens
GRFGAALTVLGDVNGDKLTDVAIGAP

SEQ ID NO: 3 - protein, 26 aa, Homo sapiens
QYFGQSLSGGQDLTMDGLVDLTVGAQ

SEQ ID NO: 4 - protein, 26 aa, Homo sapiens
YEQTRGGQVSVCPLPRGRARWQCDAV

SEQ ID NO: 5 - protein, 21 aa, Homo sapiens
DIAFLIDGSGSIIPHDFRRMK

SEQ ID NO: 6 - protein, 21 aa, Homo sapiens
RRMKEFVSTVMEQLKKSKTLF

SEQ ID NO: 7 - protein, 21 aa, Homo sapiens
SLMQYSEEFRIHFTFKEFQNN

SEQ ID NO: 8 - protein, 25 aa, Homo sapiens
PNPRSLVKPITQLLGRTHTATGIRK

SEQ ID NO: 9 - protein, 20 aa, Homo sapiens
RKVVRELFNITNGARKNAFK

SEQ ID NO: 10 - protein, 28 aa, Homo sapiens
FKILVVITDGEKFGDPLGYEDVIPEADR

SEQ ID NO: 11 - protein, 21 aa, Homo sapiens
REGVIRYVIGVGDAFRSEKSR

SEQ ID NO: 12 - protein, 22 aa, Homo sapiens
QELNTIASKPPRDHVFQVNNFE

SEQ ID NO: 13 - protein, 19 aa, Homo sapiens
ALKTIQNQLREKIFAIEGT

SEQ ID NO: 14 - protein, 15 aa, Homo sapiens
QTGSSSSFEHEMSQE

SEQ ID NO: 15 - protein, 8 aa, Homo sapiens
KSTRDRLR

SEQ ID NO: 16 - protein, 22 aa, Homo sapiens
FRSEKSRQELNTIASKPPRDHV

SEQ ID NO: 17 - protein, 20 aa, Homo sapiens
DGEKFGDPLGYEDVIPEADR

SEQ ID NO: 18 - protein, 12 aa, Homo sapiens
KEFQNNPNPRSL

SEQ ID NO: 19 - protein, 18 aa, Homo sapiens
GTQTGSSSSFEHEMSQEG

SEQ ID NO: 20 - protein, 23 aa, Homo sapiens
SNLRQQPQKFPEALRGCPQEDSD

SEQ ID NO: 21 - protein, 16 aa, Homo sapiens
RQNTGMWESNANVKGT

SEQ ID NO: 22 - protein, 15 aa, Homo sapiens
TSGSGISPSHSQRIA

SEQ ID NO: 23 - protein, 20 aa, Homo sapiens
NQRGSLYQCDYSTGSCEPIR

SEQ ID NO: 24 - protein, 9 aa, Homo sapiens
PRGRARWQC

SEQ ID NO: 25 - protein, 20 aa, Homo sapiens
KLSPRLQYFGQSLSGGQDLT

SEQ ID NO: 26 - protein, 12 aa, Homo sapiens
QKSTRDRLREGQ

SEQ ID NO: 27 - protein, 22 aa, Homo sapiens
SGRPHSRAVFNETKNSTRRQTQ

SEQ ID NO: 28 - protein, 16 aa, Homo sapiens
CETLKLQLPNCIEDPV

SEQ ID NO: 29 - protein, 15 aa, Homo sapiens
FEKNCGNDNICQDDL

SEQ ID NO: 30 - protein, 12 aa, Homo sapiens
VRNDGEDSYRTQ

SEQ ID NO: 31 - protein, 16 aa, Homo sapiens
SYRKVSTLQNQRSQRS

SEQ ID NO: 32 - protein, 9 aa, Homo sapiens
DIAFLIDGS

SEQ ID NO: 33 - protein, 9 aa, Homo sapiens
FRRMKEFVS

SEQ ID NO: 34 - protein, 11 aa, Homo sapiens
FKILVVITDGE

SEQ ID NO: 35 - protein, 11 aa, Homo sapiens
VIRYVIGVGDA

SEQ ID NO: 36 - protein, 10 aa, Homo sapiens
DGEKFGDPLG

SEQ ID NO: 37 - protein, 10 aa, Homo sapiens
YEDVIPEADR

SEQ ID NO: 38 - protein, 27 aa, Homo sapiens
YYEQTRGGQVSVCPLPRGRARWQCDAV

SEQ ID NO: 39 - DNA, 5138 bp, Homo sapiens
  CDS (95)...(3604)
gaattccctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggcatgaccttggcaacagaccccacagatggaagcattttggcctgtgaccctgggctgtctcgaacgtgtgaccagaacacctatctgagtggcctgtgttacctcttccgccagaatctgcagggtcccatgctgcaggggcgccctggttttcaggaatgtatcaagggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaatggaaggaccctgatgctctgctgaagcatgtaaagcacatgttgctgttgacaaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcctggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctctacccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgcagggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccagcctgtgaggtgcagagtgcccagaactggactcaggatgcccagggccacttcgcctctgcctgcattctgccgtgtgccctcgggcgagtcactgcctctccctggccctcagtttccctatctcgaacatggaactcattcctgaatgtctcctttgcaggctcatagggaagacctgctgagggaccagccaagagggctgcaaaagtgagggcttgtcattaccagacggttcaccagcctctcttggttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgttggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgcccacccctccttccttgaccagcagatcccagctcacgtcacacacttggttgggtcctcacatctttcacacttccaccaccctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgacgccgcatgatatgtcagggcgtgggacatctagtaggtgcttgacataatttcactgaattaatgacagagccagtgggaagatacagaaaaagagggccggggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccggacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaa

SEQ ID NO: 40 - DNA, 3533 bp, Homo sapiens
  CDS (75)...(3530)
tggcttccttgtggttcctcagtggtgcctgcaacccctggttcacctccttccaggttctggcccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaagtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagtctaacgtgacagtgactgtgagaaatgatggtgaggactcctacaggacacaggtcaccttcttcttcccgcttgacctgtcctaccggaaggtgtccacactccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcccccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtag

SEQ ID NO: 41 - DNA, 2291 bp, Homo sapiens
ctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacggaaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccatctcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggtccttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagca

SEQ ID NO: 42 - protein, 1170 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLYPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 43 - protein, 1152 aa, Homo sapiens
  SIGNAL -16 to -1
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPRESNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSPPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 44 - DNA, 4704 bp, Homo sapiens
gaattcctgccactcttcctgcaacggcccaggagctcagagctccacatctgaccttctagtcatgaccaggaccagggcagcactcctcctgttcacagccttagcaacttctctaggtttcaacttggacacagaggagctgacagccttccgtgtggacagcgctgggtttggagacagcgtggtccagtatgccaactcctgggtggtggttggagccccccaaaagataacagctgccaaccaaacgggtggcctctaccagtgtggctacagcactggtgcctgtgagcccatcggcctgcaggtgcccccggaggccgtgaacatgtccctgggcctgtccctggcgtctaccaccagcccttcccagctgctggcctgcggccccaccgtgcaccacgagtgcgggaggaacatgtacctcaccggactctgcttcctcctgggccccacccagctcacccagaggctcccggtgtccaggcaggagtgcccaagacaggagcaggacattgtgttcctgatcgatggctcaggcagcatctcctcccgcaactttgccacgatgatgaacttcgtgagagctgtgataagccagttccagagacccagcacccagttttccctgatgcagttctccaacaaattccaaacacacttcactttcgaggaattcaggcgcacgtcaaaccccctcagcctgttggcttctgttcaccagctgcaagggtttacatacacggccaccgccatccaaaatgtcgtgcaccgattgttccatgcctcatatggggcccgtagggatgccaccaaaattctcattgtcatcactgatgggaagaaagaaggcgacagcctggattataaggatgtcatccccatggctgatgcagcaggcatcatccgctatgcaattggggttggattagcttttcaaaacagaaattcttggaaagaattaaatgacattgcatcgaagccctcccaggaacacatatttaaagtggaggactttgatgctctgaaagatattcaaaaccaactgaaggagaagatctttgccattgagggtacggagaccacaagcagtagctccttcgaattggagatggcacaggagggcttcagcgctgtgttcacacctgatggccccgttctgggggctgtggggagcttcacctggtctggaggtgccttcctgtaccccccaaatatgagccctaccttcatcaacatgtctcaggagaatgtggacatgagggactcttacctgggttactccaccgagctggccctctggaaaggggtgcagagcctggtcctgggggccccccgctaccagcacaccgggaaggctgtcatcttcacccaggtgtccaggcaatggaggatgaaggccgaagtcacggggactcagatcggctcctacttcggggcctccctctgctccgtggacgtagacaccgacggcagcaccgacctggtcctcatcggggccccccattactacgagcagacccgagggggccaggtgtctgtgtgtcccttgcccagggggtggagaaggtggtggtgtgatgctgttctctacggggagcagggccacccctggggtcgctttggggcggctctgacagtgctgggggatgtgaatggggacaagctgacagacgtggtcatcggggccccaggagaggaggagaaccggggtgctgtctacctgtttcacggagtcttgggacccagcatcagcccctcccacagccagcggatcgcgggctcccagctctcctccaggctgcagtattttgggcaggcactgagcgggggtcaagacctcacccaggatggactggtggacctggctgtgggggcccggggccaggtgctcctgctcaggaccagacctgtgctctgggtgggggtgagcatgcagttcatacctgccgagatccccaggtctgcgtttgagtgtcgggagcaggtggtctctgagcagaccctggtacagtccaacatctgcctttacattgacaaacgttctaagaacctgcttgggagccgtgacctccaaagctctgtgaccttggacctggccctcgaccctggccgcctgagtccccgtgccaccttccaggaaacaaagaaccggagtctgagccgagtccgagtcctcgggctgaaggcacactgtgaaaacttcaacctgctgctcccgagctgcgtggaggactctgtgacccccattaccttgcgtctgaacttcacgctggtgggcaagcccctccttgccttcagaaacctgcggcctatgctggccgcactggctcagagatacttcacggcctccctaccctttgagaagaactgtggagccgaccatatctgccaggacaatctcggcatctccttcagcttcccaggcttgaagtccctgctggtggggagtaacctggagctgaacgcagaagtgatggtgtggaatgacggggaagactcctacggaaccaccatcaccttctcccaccccgcaggactgtcctaccgctacgtggcagagggccagaaacaagggcagctgcgttccctgcacctgacatgtgacagcgccccagttgggagccagggcacctggagcaccagctgcagaatcaaccacctcatcttccgtggcggcgcccagatcaccttcttggctacctttgacgtctcccccaaggctgtcctgggagaccggctgcttctgacagccaatgtgagcagtgagaacaacactcccaggaccagcaagaccaccttccagctggagctcccggtgaagtatgctgtctacactgtggttagcagccacgaacaattcaccaaatacctcaacttctcagagtctgaggagaaggaaagccatgtggccatgcacagataccaggtcaataacctgggacagagggacctgcctgtcagcatcaacttctgggtgcctgtggagctgaaccaggaggctgtgtggatggatgtggaggtctcccacccccagaacccatcccttcggtgctcctcagagaaaatcgcacccccagcatctgacttcctggcgcacattcagaagaatcccgtgctggactgctccattgctggctgcctgcggttccgctgtgacgtcccctccttcagcgtccaggaggagctggatttcaccctgaagggcaacctcagctttggctgggtccgccagatattgcagaagaaggtgtcggtcgtgagtgtggctgaaattacgttcgacacatccgtgtactcccagcttccaggacaggaggcatttatgagagctcagacgacaacggtgctggagaagtacaaggtccacaaccccacccccctcatcgtaggcagctccattgggggtctgttgctgctggcactcatcacagcggtactgtacaaagttggcttcttcaagcgtcagtacaaggaaatgatggaggaggcaaatggacaaattgccccagaaaacgggacacagacccccagcccgcccagtgagaaatgatccctctttgccttggacttcttctcccgcgattttccccacttacttaccctcacctgtcaggctgacggggaggaaccactgcaccaccgagagaggctgggatgggcctgcttcctgtctttgggagaaaacgtcttgcttgggaaggggcctttgtcttgtcaaggttccaactggaaacccttaggacagggtccctgctgtgttccccaaaaggacttgacttgcaatttctacctagaaatacatggacaatacccccaggcctcagtctcccttctcccatgaggcacgaatgatctttctttcctttccttttttttttttttcttttctttttttttttttttgagacggagtctcgctctgtcacccaggctggagtgcaatggcgtgatctcggctcgctgcaacctccgcctcccgggttcaagtaattctgctgtctcagcctcctgcgtagctgggactacaggcacacgccacctcgcccggcccgatctttctaaaatacagttctgaatatgctgctcatccccacctgtcttcaacagctccccattaccctcaggacaatgtctgaactctccagcttcgcgtgagaagtccccttccatcccagagggtgggcttcagggcgcacagcatgagagcctctgtgcccccatcaccctcgtttccagtgaattagtgtcatgtcagcatcagctcagggcttcatcgtggggctctcagttccgattccccaggctgaattgggagtgagatgcctgcatgctgggttctgcacagctggcctcccgcggttgggtcaacattgctggcctggaagggaggagcgccctctagggagggacatggccccggtgcggctgcagctcaccagccccaggggcagaagagacccaaccacttcctattttttgaggctatgaatatagtacctgaaaaaatgccaagcactagattatttttttaaaaagcgtactttaaatgtttgtgttaatacacattaaaacatcgcacaaaaacgatgcatctaccgctccttgggaaataatctgaaaggtctaaaaataaaaaagccttctgtggaaaaaaaaaaaaaaaaaaaaaaaaaaaaaaaaaaaaaaaaaaaaaaaaaa

SEQ ID NO: 45 - protein, 253 aa, Homo sapiens
GAEANFMLKVHPLKKYPVDLYYLVDVSASMHNNIEKLNSVGNDLSRKMAFFSRDFRLGFGSYVDKTVSPYISIHPERIHNQCSDYNLDCMPPHGYIHVLSLTENITEFEKAVHRQKISGNIDTPEGGFDAMLQAAVCESHIGWRKEAKRLLLVMTDQTSHLALDSKLAGIVVPNDGNCHLKNNVYVKSTTMEHPSLGQLSEKLIDNNINVIFAVQGKQFHWYKDLLPLLPGTIAGEIESKAANLNNLVVEAYQ

SEQ ID NO: 46 - protein, 9 aa, Homo sapiens
DVDSNGSTD

SEQ ID NO: 47 - protein, 9 aa, Homo sapiens
DVNGDKLTD

SEQ ID NO: 48 - protein, 9 aa, Homo sapiens
DLTMDGLVD

SEQ ID NO: 49 - protein, 9 aa, Homo sapiens
DSDMNDAYL

SEQ ID NO: 50 - protein, 33 aa, Homo sapiens
NAFKILVVITDGEKFGDPLGYEDVIPEADREGV

SEQ ID NO: 51 - protein, 5 aa, Homo sapiens
DGEKF

SEQ ID NO: 52 - protein, 253 aa, Homo sapiens
GEPQQLQVRFLRAEGYPVDLYYLMDLSSSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGSCHLDSNGLYSRSTEFDYPSVGQVAQALSAANIQPIFAVTSAALPVYQELSKLIPKSAVGELSEDSSNVVQLIMDAYN

SEQ ID NO: 53 - protein, 255 aa, Homo sapiens
GGAQTLQVHVRQTEDYPVDLYYLMDLSASMDDDLNTIKELGSGLSKEMSKLTSNFRLGFGSFVEKPVSPFVKTTPEEIANPCSSIPYFCLPTFGFKHILPLTNDAERFNEIVKNQKISANIDTPEGGFDAIMQAAVCKEKIGWWRNDSLHLLVFVSDADSHFGMDSKLAGIVIPNDGLCHLDSKNEYSMSTVLEYPTIGGLIDKLVQNNVLLIFAVTQEQVHLYENYAKLIPGATVGLLQKDSGNILQLIISAYE

SEQ ID NO: 54 - protein, 256 aa, Homo sapiens
GDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYN

SEQ ID NO: 55 - protein, 252 aa, Homo sapiens
GEERHFELEVFEPLESPVDLYILMDFSNSMSDDLDNLKKMGQNLARVLSQLTSDYTIGFGKFVDKVSVPQTDMRPEKLKEPWPNSDPPFSFKNVISLTEDVDEFRNKLQGERISGNLDAPEGGFDAILQTAVCTRDIGWRPDSTHLLVFSTESAFHYEADGANVLAGIMSRNDERCHLDTTGTYTQYRTQDYPSVPTLVRLLAKHNIIPIFAVTNYSYSYYEKLHTYFPVSSLGVLQEDSSNIVELLEEAFN

SEQ ID NO: 56 - protein, 255 aa, Homo sapiens
DDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYG

SEQ ID NO: 57 - protein, 252 aa, Homo sapiens
GQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYN

SEQ ID NO: 58 - protein, 251 aa, Homo sapiens
GEPQTFTLKFKRAEDYPIDLYYLMDLSYSMKDDLENVKSLGTDLMNEMRRITSDFRIGFGSFVEKTVMPYISTTPAKLRNPCTSEQNCTTPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYN

SEQ ID NO: 59 - protein, 187 aa, Homo sapiens
CPRQEQDIVFLIDGSGSISSRNFATMMNFVRAVISQFQRPSTQFSLMQFSNKFQTHFTFEEFRRTSNPLSLLASVHQLQGFTYTATAIQNVVHRLFHASYGARRDATKILIVITDGKKEGDSLDYKDVIPMADAAGIIRYAIGVGLAFQNRNSWKELNDIASKPSQEHIFKVEDFDALKDIQNQLKE

SEQ ID NO: 60 - protein, 181 aa, Homo sapiens
CIKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFFEKLKDLFIERQK

SEQ ID NO: 61 - protein, 20 aa, Homo sapiens
CPQEDSDIAFLIDGSGSIIP

SEQ ID NO: 62 - protein, 20 aa, Homo sapiens
IIPHDFRRMKEFVSTVMEQL

SEQ ID NO: 63 - protein, 20 aa, Homo sapiens
EQLKKSKTLFSLMQYSEEFR

SEQ ID NO: 64 - protein, 20 aa, Homo sapiens
EFRIHFTFKEFQNNPNPRSL

SEQ ID NO: 65 - protein, 20 aa, Homo sapiens
RSLVKPITQLLGRTHTATGI

SEQ ID NO: 66 - protein, 20 aa, Homo sapiens
TGIRKVVRELFNITNGARKN

SEQ ID NO: 67 - protein, 29 aa, Homo sapiens
KVVRELSNITNGARKNASKILVVITDGEK

SEQ ID NO: 68 - protein, 17 aa, Homo sapiens
DREGVIRYVIGVGDAFR

SEQ ID NO: 69 - protein, 20 aa, Homo sapiens
HVFQVNNFEALKTIQNQLRE

SEQ ID NO: 70 - protein, 14 aa, Homo sapiens
NAFKILVVITDGEK

SEQ ID NO: 71 - protein, 7 aa, Homo sapiens
NAFKILV

SEQ ID NO: 72 - protein, 7 aa, Homo sapiens
VITDGEK

(19) United States
(12) Patent Application Publication
(10) Pub. No.: US 2005/0227296 A1
(43) Pub. Date: Oct. 13, 2005

(54) Method for indentifying integrin antagonists

(75) Inventor: M. Amin Arnaout, Chestnut Hill, MA (US)

Correspondence Address: FISH & RICHARDSON PC, P.O. BOX 1022, MINNEAPOLIS, MN, 55440-1022, US

(73) Assignee: The General Hospital Corporation, a Massachusetts corporation

(21) Appl. No.: 11/000,473

(22) Filed: Nov. 30, 2004

Related U.S. Application Data
(63) Continuation of application No. 10/802,508, filed on Mar. 17, 2004, now abandoned.
Application No. 10/802,508 is a continuation of application No. 09/592,617, filed on Jun. 13, 2000, now abandoned.
Application No. 09/592,617 is a continuation of application No. 08/380,167, filed on Jan. 30, 1995, now abandoned.
Application No. 08/380,167 is a continuation-in-part of application No. 08/216,081, filed on Mar. 21, 1994, now abandoned.
Application No. 08/216,081 is a continuation of application No. 07/637,830, filed on Jan. 4, 1991, now abandoned.
Application No. 07/637,830 is a continuation-in-part of application No. 07/539,842, filed on Jun. 18, 1990, now abandoned.
Application No. 07/539,842 is a continuation-in-part of application No. 07/212,573, filed on Jun. 28, 1988, now abandoned.

(51) Int. Cl. G01N 33/53; G01N 33/567

(52) U.S. Cl. 435/7.2

(57) ABSTRACT

The invention features methods for identifying compounds capable of inhibiting the binding of a selected integrin to a selected ligand which naturally binds the selected integrin.

### CROSS REFERENCE TO RELATED APPLICATIONS

This application is a continuation-in-part of co-pending application U.S. Ser. No. 08/216,081, filed Mar. 21, 1994, which in turn is a continuation-in-part of my earlier, co-pending application U.S. Ser. No. 539,842, filed Jun. 18, 1990, which is in turn a continuation-in-part of my earlier application U.S. Ser. No. 212,573, filed Jun. 28, 1988, now abandoned, both of which are hereby incorporated by reference.

### BACKGROUND OF THE INVENTION

This invention relates to methods for identifying molecules capable of interfering with certain cellular immune/inflammatory responses, particularly phagocyte-mediated tissue injury and inflammation.

Circulating phagocytic white blood cells are an important component of the cellular acute inflammatory response. It is believed that a number of important biological functions such as chemotaxis, immune adherence (homotypic cell adhesion or aggregation), adhesion to endothelium, phagocytosis, antibody-dependent cellular cytotoxicity, superoxide, and lysosomal enzyme release are mediated by a family of leukocyte surface glycoprotein adhesion receptors known as β₂ integrins or the CD11/CD18 complex. Arnaout et al., Blood 75:1037 (1990).

The CD11/CD18 family consists of four heterodimeric surface glycoproteins, each with a distinct a subunit (CD11a, CD11b, CD11c, or CD11d) non-covalently associated with a common 8 subunit (CD18). The divalent cations Ca⁺² and Mg²⁺ are essential in the stabilization and function of the αβ complex.

The CD11/CD18 integrins mediate the stable adhesion of leukocytes to endothelium and the subsequent ransendothelial migration into inflamed organs (Hynes, Cell 69:11, 1992). CD11b/CD18 also mediates aggregation of phagocytes (Arnaout et al., N. Engl. J. Med. 312:457, 1985), ingestion of opsonized particles, and the generation of oxygen free radicals and release of hydrolytic enzymes in response to particulate stimuli (Arnaout et al., J. Clin. Invest. 72:171, 1983). Inherited deficiency of CD11/CD18 integrins (Leu-CAM deficiency, LAD) results in life-threatening pyogenic infections and poor wound healing due to the inability of circulating phagocytes to extravasate into infected tissues and to clear pathogens through phagocytosis and cell-mediated killing (Arnaout, Immunol. Rev. 114:145, 1990).

While essential for host survival, CD11/CD18 integrin-mediated influx and inflammatory functions in phagocytes often exacerbate the local pathologic lesions and tissue injury in many noninfectious disease states including hemorrhagic shock, burns, atherosclerosis and hyperacute rejection (Albeda et al., FASEB J. 8:504, 1994). In several animal models of inflammation, monoclonal antibodies to CD11b/CD18 and other CD11/CD18 integrins markedly reduce the influx and inflammatory functions of leukocytes, thus preserving tissue integrity and host survival.

The functions of CD11b/CD18 in leukocyte extravasation and inflammation are mediated through its binding to several physiologic ligands, including iC3b, the major complement C3 opsonin (Wright et al., Proc. Nat'l Acad. Sci. 80:5699, 1983), CD54 (intercellular adhesion molecule-1, ICAM-1 (Simmons et al., Nature 331:625, 1988), and the coagulation factors fibrinogen and factor X (Altieri et al., J. Cell. Biol. 107:1893, 1988).

### SUMMARY OF THE INVENTION

The invention features methods for identifying antagonists of integrin function. The methods entail the use of an A-domain peptide, or ligand binding fragment thereof, derived from CD11b, CD11a, CD11c, CD18 (also known as β2) or any of the integrin β subunits having an A-domain (e.g., β1, β3, β4, β5, β6, β7, and β8).

In one aspect, the invention features an in vitro method of screening candidate compounds for the ability to inhibit the binding of a selected integrin to a selected ligand which naturally binds to the selected integrin, the method includes:
a) measuring the binding of an A-domain peptide derived from the selected integrin to the selected ligand in the presence of the candidate compound;
b) measuring the binding of the A-domain peptide derived from the selected integrin to the selected ligand in the absence of the candidate compound;
c) determining whether the binding is decreased in the presence of the candidate compound;
d) identifying inhibiting compounds as those which decrease the binding.

In a preferred embodiment the selected integrin is a β2 integrin. In more preferred embodiments the β2 integrin is selected from the group comprising CD11a/CD18, CD11b/CD18, and CD11c/CD18; the β2 integrin is CD11b/CD18; the β2 integrin is CD11a/CD18; the β2 integrin is CD11c/CD18.

In another preferred embodiment the method of claim 2 wherein the A-domain peptide is derived from the α subunit of the selected integrin; the A-domain peptide is a CD11b A-domain peptide; the A-domain peptide is a CD11a A-domain peptide; the A-domain peptide is a CD11c A-domain peptide; the A-domain peptide is derived from the B subunit of the elected integrin; the ligand is detectably labelled.

In another aspect the invention features an in vitro method of screening candidate compounds for the ability to bind to a selected integrin, the method includes:
a) measuring the binding of an A-domain peptide derived from the selected integrin to the candidate compound;
d) identifying compounds capable of binding the selected integrin as those which bind to the A-domain peptide.

In one aspect of the invention candidate antagonists (e.g., peptides, antibodies, or small molecules) are tested for their ability to bind a selected A-domain peptide (or ligand-binding portion thereof). For example, a CD11b A-domain peptide can be immobilized on a solid support and then incubated with a detectably labelled candidate antagonist. Candidate antagonists which bind to the CD11b A-domain peptide can then be further characterized by examining whether they are capable of inhibiting the interaction between the selected A domain peptide and a ligand which naturally binds to the integrin which includes the selected A domain peptide. Thus, a candidate antagonist of CD11b/CD18 function identified by its ability to bind to CD11b A domain peptide can be examined to determine whether it is capable of inhibiting the binding of EAiC3b (a natural ligand of CD11b/CD18) and CD11b/CD18 (e.g., recombinant CD11b/CD18 expressed in COS cells).

In another aspect the of the invention candidate antagonists (e.g., peptides, antibodies, or small molecules) are tested for their ability to inhibit the binding of a selected A-domain peptide (or ligand-binding portion thereof) to a ligand to which the integrin from which the peptide is derived naturally binds. Candidate antagonists which inhibit such a binding interaction are very likely able to inhibit the interaction between the integrin from which the A-domain was derived and the ligand. Such candidate antagonists are thus likely to be capable of interferring with an immune response mediated by interaction between the integrin and ligand. For example, a CD11b A-domain peptide can be immobilized on a solid support and then incubated with a detectably ligand (e.g., iC3b) in the presence and absence of the candidate antagonist. If binding of the CD11b A-domain peptide to iC3b is less in the presence of the candidate antagonist than in the absence of the candidate antagonist are likely capable of inhibiting the interaction between the selected A domain peptide and a ligand which naturally binds to the integrin which includes the selected A domain peptide.

In either case, the candidate ligands identified by the method of the invention can be furhter characterized using any of the in vitro and in vivo assays described herein or known to those skilled in the art.

Ligands of CD11a/CD18 include: ICAM-1, ICAM-2, ICAM-3. Ligands of CD11b/CD18 and CD11c/CD18 include: ICAM-1, ICAM-2, iC3b, fibrinogen, NIF, LPS, gp63, CD23, and other endothelial, epithelial, and neutrophil ligands. Ohter lignads of CD11b and other integrins are shown in FIG. 9.

In the method of the invention the ligand need not be an isolated protein. For example cells which express the ligand or have the ligand present on their surface can be used in the screening methods of the invention.

Molecules which antagonize one or more integrin-mediated immune responses can be useful in therapeutic interventions of inflammatory diseases.

By “ligand which naturally binds to a integrin” is meant a molecule, often a protein, whihc binds to the integrin in the course of a normally occuring cell-cell, cell-matrix, or matrix-matrix interaction.

By “derived from” an integrin is meant that the A-domain is found within that integrin.

By “A-domain peptide” is meant a sequence designated herein as an A-domain or an amino acid sequence produced by introducing one or more conservative amino acid substitutions in an amino acid sequence corresponding to the sequence corresponding to that sequence. By “naturally occuring A-domain peptide” is meant a peptide sequence designated herein as an A-domain sequence. By “ligand-binding fragment” of an A-domain peptide is meant a streach of at least 10, preferably at least 20, 30, 50, or 100 amino acids within an “A-domain peptide” which retains the ability, under standard assay condition, to bind a “ligand which naturally binds to a integrin” from which the A-domain peptide is derived.

“β2 integrins” and “CD11/CD18” include all leukocyte adhesion molecules which include a CD18 subunit. By the “A domain of CD11b” is meant the amino acid sequence of CD11b from Cys¹²⁸ to Glu³²¹ or an amino acid sequence produced by introducing one or more conservative amino acid substitutions in an amino acid sequence corresponding to the sequence of CD11b from Cys¹²⁸ to Glu³²¹. “CD11/CD18-mediated immune response” includes those CD11/CD18-related functions mentioned above: chemotaxis, immune adherence (homotypic cell adhesion or aggregation), adhesion to endothelium, phagocytosis, antibody-dependent or antibody-independent cellular cytotoxicity, and superoxide and lysosomal enzyme release. Inhibition of these immune functions can be determined by one or more of the following inhibition assays as described in greater detail below: iC3b binding, cell-cell aggregation, phagocytosis, adhesion to endothelium, and chemotaxis. As used herein, a human CD11b recombinant peptide is a chain of amino acids derived from recombinant CD11b-encoding cDNA, or the corresponding synthetic DNA.

By “polypeptide” is meant any chain of amino acids, regardless of length or post-translational modification (e.g., glycosylation or phosphorylation).

By “substantially identical” is meant a polypeptide or nucleic acid exhibiting at least 50%, preferably 85%, more preferably 90%, and most preferably 95% homology to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

Sequence identity is typically measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications.

Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine.

By a “substantially pure polypeptide” is meant a polypeptide which has been separated from components which naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, Rps2 polypeptide. A substantially pure CD11 or CD18 polypeptide may be obtained, for example, by extraction from a natural source (e.g., a human leukocyte); by expression of a recombinant nucleic acid encoding a CD11 or CD18 polypeptide; or by chemical synthesis. Purity can be measured by any appropriate method, e.g., those described in column chromatography, polyacrylamide gel electrophoresis, or by HPLC analysis.

A polypeptide or protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes.

By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

By “transformed cell” is meant a cell into which (or into an ancestor of which) has been introduced, by means of recombinant. DNA techniques, a DNA molecule encoding (as used herein) polypeptide (e.g., a CD11b or CD18 polypeptide).

By “peptide homologous to an A-domain peptide” is meant any peptide of 15 or more contiguous amino acids exhibiting at least 30%, preferably 50%, and most preferably 70% amino acid sequence identity to the A-domain of CD11b.

By “detectably-labelled” is meant any means for marking and identifying the presence of a molecule, e.g., an oligonucleotide probe or primer, a gene or fragment thereof, or a cDNA molecule. Methods for detectably-labelling a molecule are well known in the art and include, without limitation, radioactive labelling (e.g., with an isotope such as ³²P or ³⁵S) and nonradioactive labelling (e.g., chemiluminescent labelling, e.g., fluorescein labelling).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody, e.g., an CD11b A domain-specific antibody. A purified CD11b A domain antibody may be obtained, for example, by affinity chromatography using recombinantly-produced CD11b A domain polypeptide and standard techniques.

By “specifically binds” is meant an antibody which recognizes and binds an rps protein but which does not substantially recognize and bind other molecules in a sample, e.g., a biological sample, which naturally includes rps protein.

The peptides and heterodimeric proteins of the invention are capable of antagonizing CD11/CD18 (82 integrin) mediated immune response. CD11/CD18 mediated immune responses which it may be desirable to block include acute inflammatory functions mediated by neutrophils. The molecules of the invention are useful for treatment of ischemia reperfusion injury (e.g., in the heart, brain, skin, liver or gastrointestinal tract), burns, frostbite, acute arthritis, asthema, and adult respiratory distress syndrome. Peptides and heterodimeric proteins of the invention may also be useful for blocking intra-islet infiltration of macrophages associated with insulin-dependent diabetes mellitus.

The invention features a purified peptide which includes at least one extracellular region of a β2 integrin subunit capable of inhibiting a CD11/CD18 mediated immune response, the peptide lacks the transmembrane and cytoplasmic portions of the 82 integrin subunit. In a preferred embodiment the β2 integrin subunit is a human β2 integrin subunit; more preferably the β2 integrin subunit is CD11a, CD11b, CD11c or CD18; most preferably the β2 integrin subunit is CD11b. Preferably, the peptide includes all or part of the A domain of CD11b. More preferably the peptide includes one of the following sequences: DIAFLIDGS (SEQ ID NO: 32); FRRMKEFVS (SEQ ID NO: 33); FKILVVITDGE (SEQ ID NO: 34); VIRYVIGVGDA (SEQ ID NO: 35); DGEKFGDPLG (SEQ ID NO: 36); YEDVIPEADR (SEQ ID NO: 37); DGEKFGDPLGYEDVIPEADR (SEQ ID NO: 17); NAFKILVVITDGEKFGDPLGYEDVIPEADREGV (SEQ ID NO: 50); DGEKF (SEQ ID NO: 51). In preferred embodiments, the peptide includes the amino acid sequence YYEQTRGGQVSVCPLPRGRARWQCDAV (SEQ ID NO: 38); the peptide includes the amino acid sequence KSTRDRLR (SEQ ID NO: 15). Preferably, the peptide includes one of the following amino acid sequences: AYFGASLCSVDVDSNGSTDLVLIGAP (SEQ ID NO: 1); GRFGAALTVLGDVNGDKLLTDVAIGAP (SEQ ID NO: 2); QYFGQSLSGGQDLTMDGLVDLTVGAQ (SEQ ID NO: 3); YEQTRGGQVSVCPLPRGRARWQCDAV (SEQ ID NO: 4); DIAFLIDGSGSIIPHDFRRMK (SEQ ID NO: 5); RRMKEFVSTVMEQLKKSKTLF (SEQ ID NO: 6); SLMQYSEEFRIHFTFKEFQNN (SEQ ID NO: 7); PNPRSLVKPITQLLGRTHTATGIRK (SEQ ID NO: 8); RKVVRELFNITNGARKNAFK (SEQ ID NO: 9); FKILVVITDGEKFGDPLGYEDVIPEADR (SEQ ID NO: 10); REGVIRYVIGVGDAFRSEKSR (SEQ ID NO: 11); QELNTIASKPPRDHVFQVNNFE (SEQ ID NO: 12); ALKTIQNQLREKIFAIEGT (SEQ ID NO: 13); QTGSSSSFEHEMSQE (SEQ ID NO: 14); FRSEKSRQELNTIASKPPRDHV (SEQ ID NO: 16); KEFQNNPNPRSL (SEQ ID NO: 18); GTQTGSSSSFEHEMSQEG (SEQ ID NO: 19); SNLRQQPQKFPEALRGCPQEDSD (SEQ ID NO: 20); RQNTGMWESNANVKGT (SEQ ID NO: 21); TSGSGISPSHSQRIA (SEQ ID NO: 22); NQRGSLYQCDYSTGSCEPIR (SEQ ID NO: 23); PRGRARWQC (SEQ ID NO: 24); KLSPRLQYFGQSLSGGQDLT (SEQ ID NO: 25); QKSTRDRLREGQ (SEQ ID NO: 26); SGRPHSRAVFNETKNSTRRQTQ (SEQ ID NO: 27); CETLKLQLPNCIEDPV (SEQ ID NO: 28); FEKNCGNDNICQDDL (SEQ ID NO: 29); VRNDGEDSYRTQ (SEQ ID NO: 30); SYRKVSTLQNQRSQRS (SEQ ID NO: 31).

Preferably, the peptide includes one or more metal binding domains of CD11b. More preferably, the metal binding domains encompass amino acids 358-412, 426-483, 487-553, and 554-614 of CD11b. Most preferably, the peptide includes one of the following sequences: DVDSNGSTD (SEQ ID NO: 46); DVNGDKLTD (SEQ ID NO: 47); DLTMDGLVD (SEQ ID NO: 48); DSDMNDAYL (SEQ ID NO: 49).

In a preferred embodiment, the peptides are soluble under physiological conditions.

In another aspect, the invention features a method of controlling phagocyte-mediated tissue damage to a human patient. The method includes administering a therapeutic composition to a patient; the therapeutic composition includes a physiologically acceptable carrier and a peptide or a heterodimer of the invention. More preferably, the method is used to control phagocyte-mediated tissue damage due to ischemia-reperfussion. Most preferably, the method is used to control phagocyte-mediated tissue damage to the heart muscle associated with reduced perfusion of heart tissue during acute cardiac insufficiency.

In another aspect, the invention features a monoclonal antibody which is raised to a peptide or a heterodimer of the invention and which is capable of inhibiting a CD11/CD18 mediated immune response.

In another aspect, the features a human CD11b recombinant peptide.

“CD11¹⁰⁸⁹/CD¹⁸⁶⁹⁹” is a heterodimer which comprises amino acids 1-1089 of human CD11 and amino acids 1-699 of CD18.

Other features and advantages of the invention will be apparent from the following description of the preferred embodiments thereof, and from the claims.

### DESCRIPTION OF THE PREFERRED EMBODIMENTS

The drawings will first briefly be described.

### DRAWINGS

FIG. 1 is the cDNA sequence and deduced amino acid sequence of the open reading frame of human CD11b from Arnaout et al., J. Cell. Biol. 106:2153 (1988).

FIG. 2 is a representation of the results of an immunoprecipitation assay.

FIG. 3 is a representation of the results of an immunoprecipitation assay.

FIG. 4 is a representation of the results of an immunoprecipitation assay.

FIG. 5 is a graph of the effect of various proteins and antibodies on neutrophil adhesion to endothelium.

FIG. 6 is the cDNA sequence and deduced amino acid sequence of human CD11a from Larson et al., J. Cell. Biol. 108:703 (1989).

FIG. 7 is the cDNA sequence and deduced amino acid sequence of human CD11c from Corbi et al., EMBO J. 6:4023 (1987).

FIG. 8 is the cDNA sequence of human CD18 from Law et al., EMBO J. 6:915 (1987).

FIG. 9 is a schematic illustration of some of the naturally occurring ligands for various integrins. The β subunit are boxed. The a subunits are circled. The pairing of subunits is indicated with lines drawn between the relevant α and β subunits which make up the heterodimer. In each case the some of the ligands which naturally bind the heterodimer are indicated above the line, and its tissue distribution is indicated below the line in italics. (Co=collagens; LM=laminin; FN=fibronectin; VN=vitronectin; TSP=thrombospondin; FB=fibrinogen; vWf=von Willebrand factor; OP=osteopontin; FX=factor X; CHO=carbohydrates; BSP1=bone sialoprotein 1; L=lymphocyte; N monocyte/macrophage; PMN=granulocytes; E=eosinophils; B=basophils; NK=natural killer cells; PLT=platelets; IEL=intestinal intraepithelial lymphocytes; PBL=peripheral blood leukocytes; L-=L-selectin negative; EPI=epithelial cells; ENDO=endothelial cells; MYO=myocytes; NEU=neural tissue; MEL=melanoma; FIB=fibroblasts).

FIG. 10 is the sequence of the A-domains of β1-88. In each case the sequences between “A” and “B” (each indicated by arrows) represent full length A-domain. A-domain fragments include: the sequences between “A” and “C” (both indicated by arrows); the sequences between “D” and “C” (both indicated by arrows); and the sequences between “D” and “B” (both indicated by arrows).

FIG. 11 is the sequences of the A-domains of CD11a and CD11c.

FIG. 12 is the sequences of certain CD11b fragments employed in certain binding experiments.

### PEPTIDES

Each member of the β2 integrin family is a heterodimer consisting of two subunits: a CD11 subunit (with at least three variants designated CD11a, CD11b, and CD11c) and a CD18 subunit. Each subunit includes a transmembrane anchor which connects a cytoplasmic segment to an extracellular segment. The two subunits interact to form a functional heterodimer. As described in greater detail below, the extracellular segments of the 82 integrin subunits contain various functional domains.

Without wishing to bind myself to a particular theory, it appears that the peptides of the invention antagonize CD11/CD18-mediated immune responses by competitively inhibiting binding of leukocytes bearing a member of the β2 integrin family to the respective binding partners of that family. Specifically, the peptides of the invention include an immune-response inhibiting extracellular segment of any one of the β₂ integrin subunits—CD11a, CD11b, CD11c, CD18—or a heterodimer composed of a portion of an α (CD11a, CD11b, or CD11c) subunit together with a portion of a β subunit (CD18). Candidate β2 integrin subunits can be evaluated for their ability to antagonize CD11/CD18-mediated immune responses by any of several techniques. For example, subunits may be tested for their ability to interfere with neutrophil adhesion to endothelial cells using an assay described in detail below. Specific regions of the β2 integrin subunits can be evaluated in a similar manner. Any extracellular region of a β2 integrin subunit may be screened for its ability to interfere with CD11/CD18 mediated immune response. Regions of CD11 whose sequences are conserved between two or more subunits are preferred candidates for antagonizing. CD11/CD18—mediated immune response. For example, the A domain (corresponding to Cys¹²⁸ to Glu³²¹ of CD11b) is conserved between CD11a, CD11b, and CD11c. The A domain is 64% identical in CD11b and CD11c and 36% homologous between these two subunits and CD11a. This domain is also homologous to a conserved domain in other proteins involved in adhesive interactions including von Willebrand's factor, cartilage matrix protein, VLA2, and the complement C3b/C4b—binding proteins C2 and factor B. The extracellular portions of CD11a, CD11b and CD11c include seven homologous tandem repeats of approximately 60 amino acids. These repeats are also conserved in the α subunits of other integrin subfamilies (e.g., fibronectin receptor). Arnaout et al., Blood 75:1037 (1990).

Regions of CD18 which are conserved among β integrin subunits (i.e., the β subunits of β1, β2 and β3 integrins) are also good candidates for regions capable of interfering with CD11/CD18-mediated immune response. For example, CD18 has four tandem repeats of an eight-cysteine motif. This cysteine-rich region is conserved among β subunits. Just amino terminal to this cysteine rich region is another conserved region, 247 amino acids long, which is conserved in several integrin β subunits.

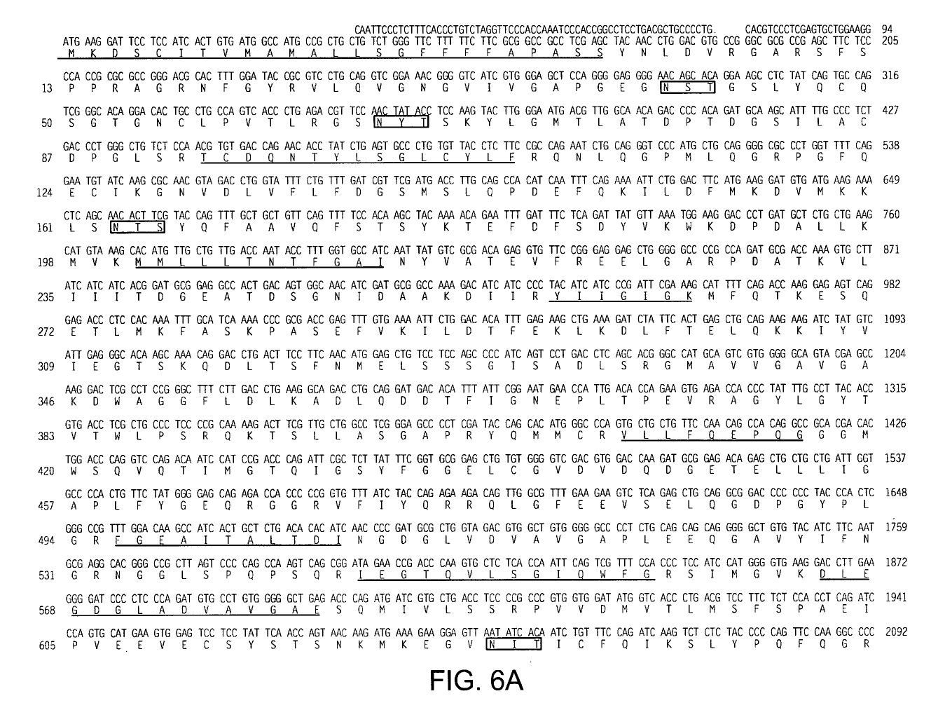

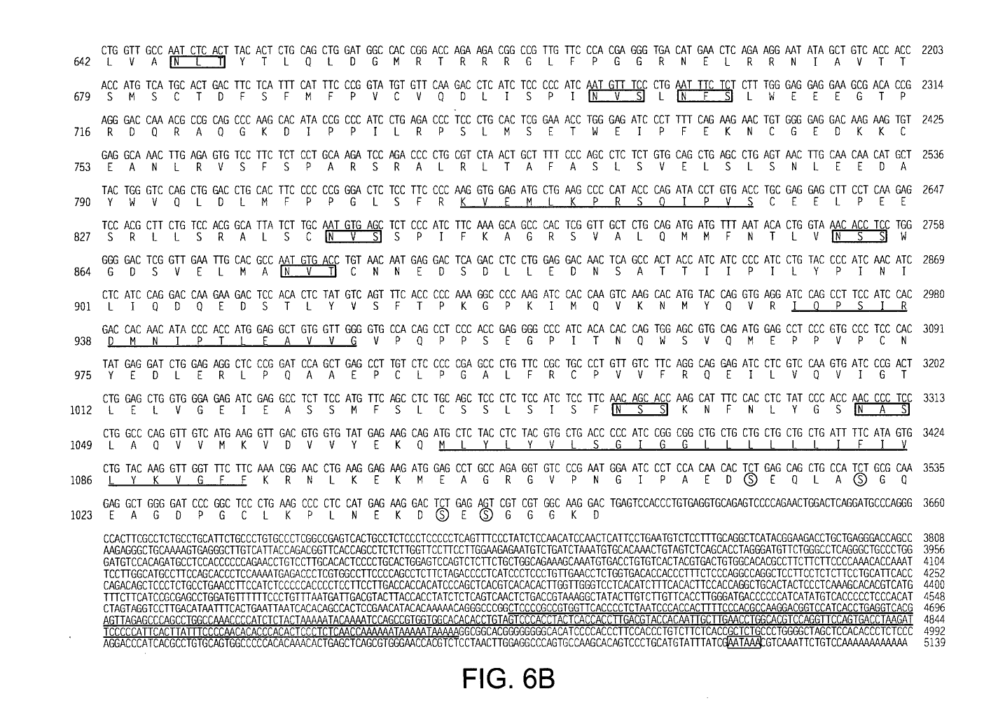

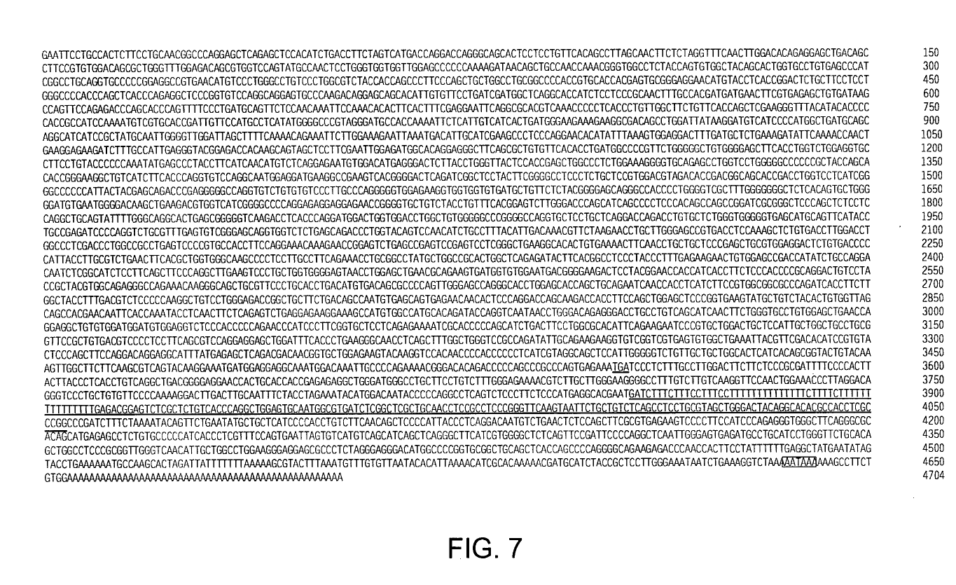

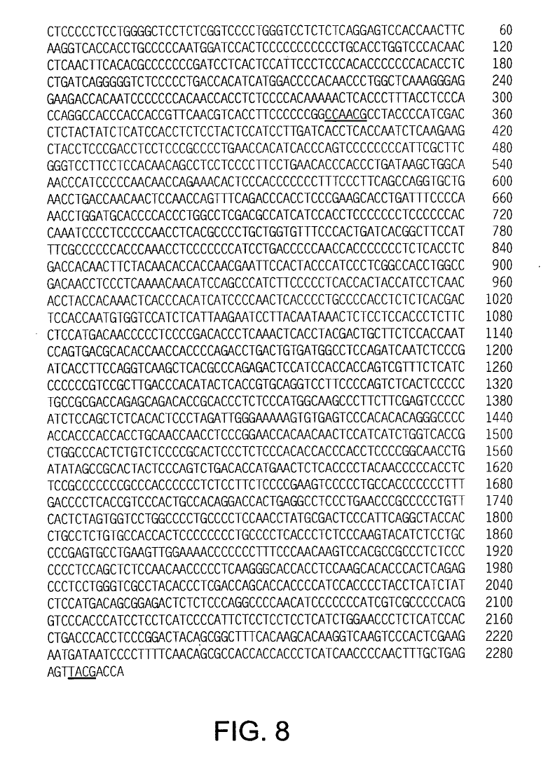

FIG. 6 depicts the cDNA sequence of human CD11a (SEQ ID NO: 39); FIG. 7 depicts the cDNA sequence of human CD11c (SEQ ID NO: ); FIG. 8 depicts the cDNA sequence of CDIS (SEQ ID NO: 41).

DNA molecules encoding all or part of CD11a, CD11b, CD11c or CD18 can be obtained by means of polymerase chain reaction amplification. In this technique two short DNA primers are used to generate multiple copies of a DNA fragment of interest from cells known to harbor the mRNA of produced by the gene of interest. This technique is described in detail by Frohman et al., Proc. Nat'l Acad Sci. USA 85:8998 (1988). Polymerase chain reaction methods are generally described by Mullis et al. (U.S. Pat. Nos. 4,683,195 and 4,683,202).

For example, to clone a portion of CD11a, the known sequence of CD11a is used to design two DNA primers which will hybridize to opposite strands outside (or just within) the region of interest. The primers must be oriented so that when they are extended by DNA polymerase, extension proceeds into the region of interest. To generate the CD11a DNA, polyA RNA is isolated from cells expressing CD11a. A first primer and reverse transcriptase are used to generate a cDNA form the mRNA. A second primer is added; and Taq DNA polymerase is used to amplify the cDNA generated in the previous step. Alternatively, the known sequences of CD11a, CD11b, CD11c and CD18 can be used to design highly specific probes for identifying cDNA clones harboring the DNA of interest. A cDNA library suitable for isolation of CD11a, CD11b, and CD11c DNA can be generated using phorbol ester-induced HL-60 cells (ATCC Accession No. CCL 240) as described by Corbi et al. (EMBO J. 6:4023, 1987) and Arnaout et al., Proc. Nat'l Acad. Sci. USA 85:2776, 1988); CD18 DNA can be isolated from a library generated using U937 cells (ATCC Accession No. CRL 1593) as described by Law et al. (EMBO J. 6:915, 1987). These cell lines are also suitable for generating cDNA by polymerase chain reaction amplification of mRNA as described above.

Isolation of a Human CD11b cDNA Clone.

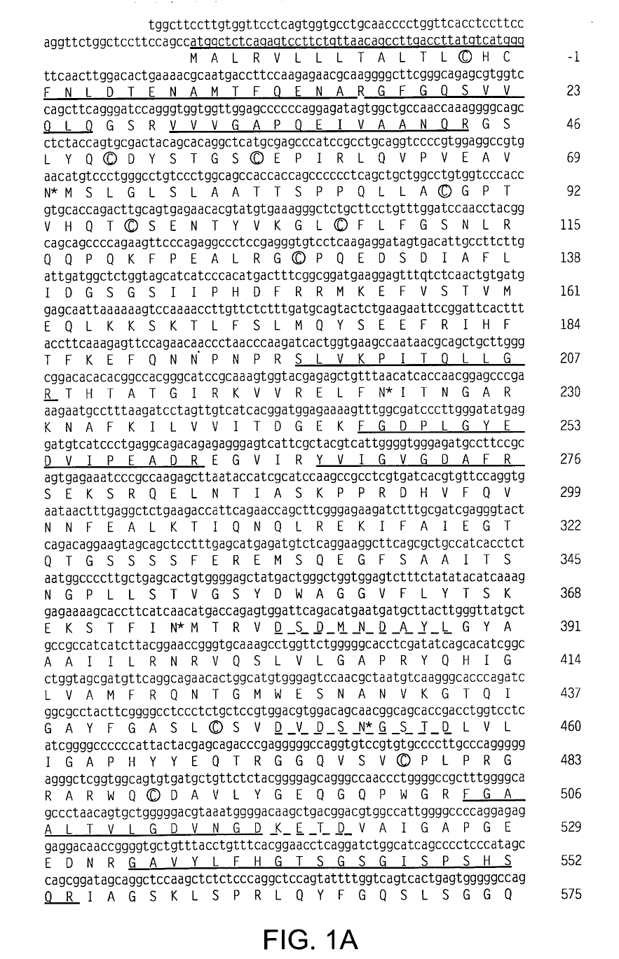

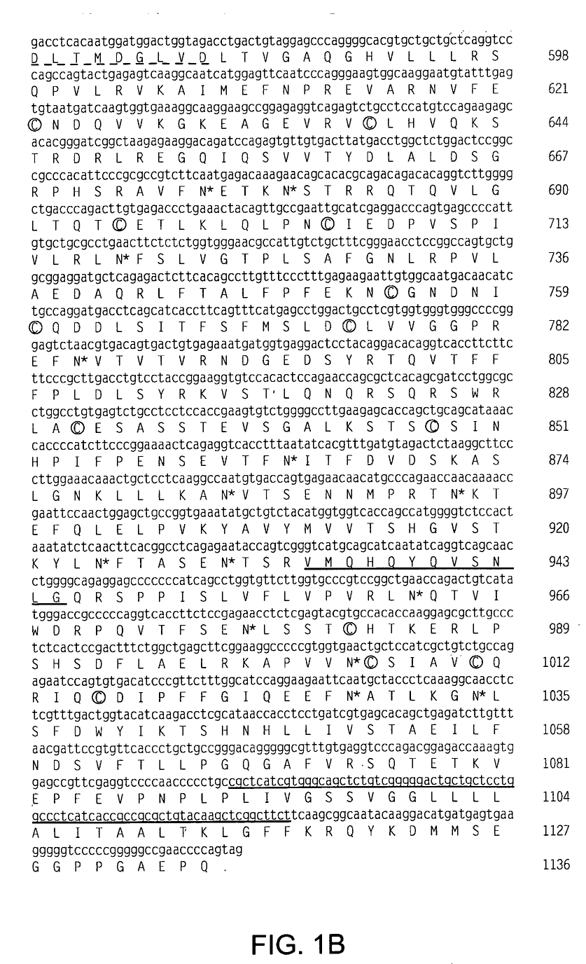

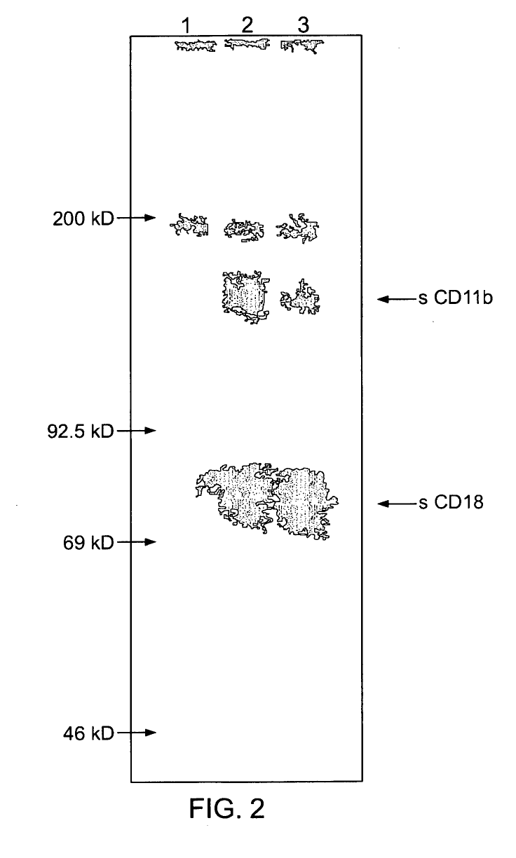

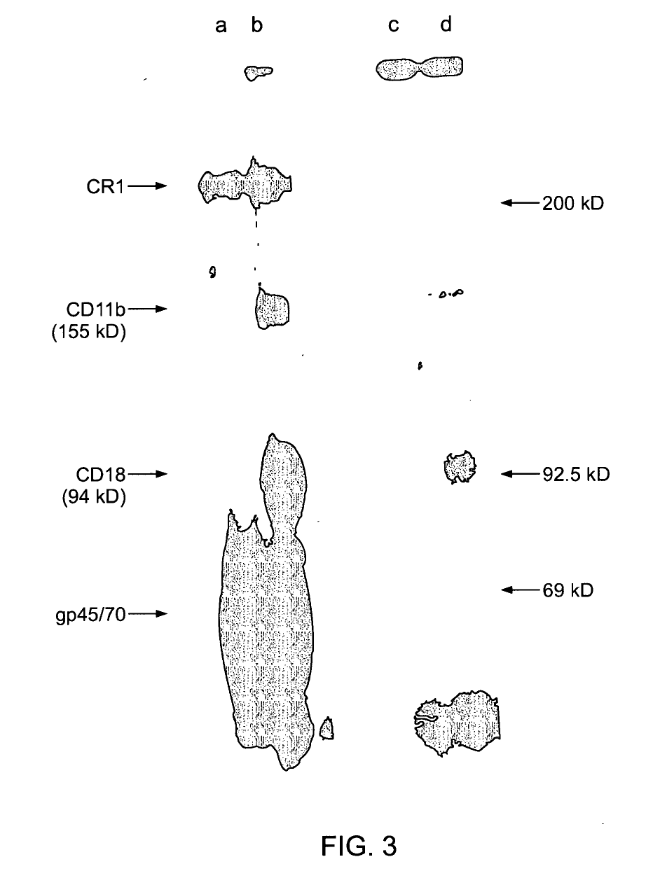

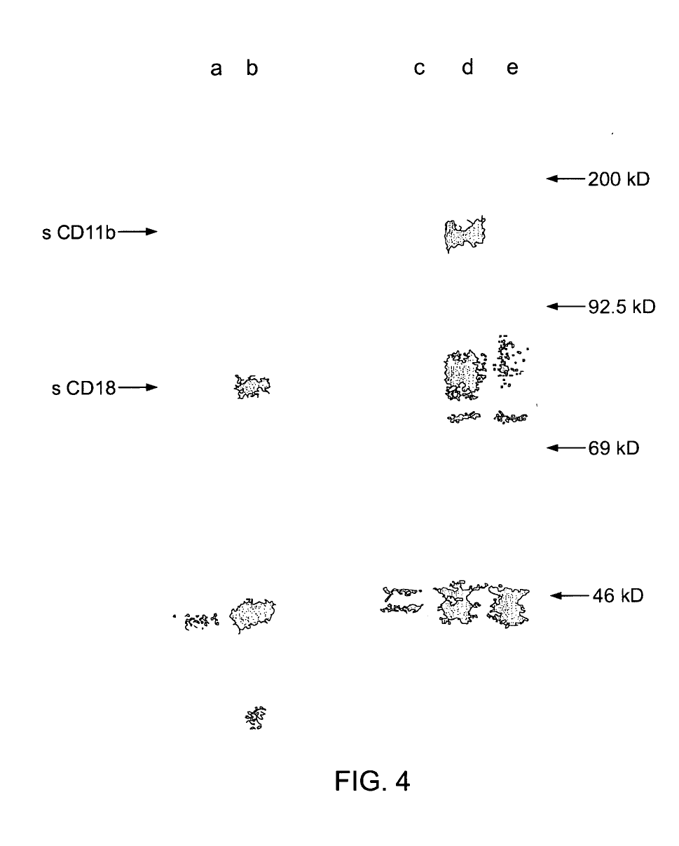

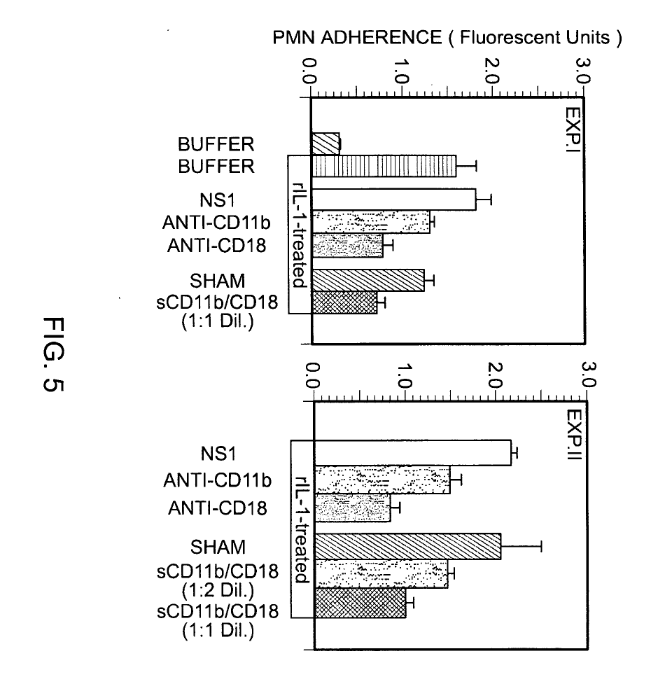

A 378 base pair (bp) cDNA clone encoding guinea pig CD11b was used as a probe to isolate three additional cDNA clones from a human monocyte/lymphocyte cDNA library as described in Arnaout et al., Proc. Nat'l. Acad. Sci. USA 85:2776 (1988); together these three clones contain the 3,048 nucleotide sequence encoding the CD11b gene shown in FIG. 1 (SEQ ID NO: 40). Arnaout et al., J. Cell. Biol. 106:2153 (1988).

In order to express CD11b, a mammalian expression vector was constructed by assembling the above-described three cDNA clones. Appropriate restriction enzyme sites within the CD11b gene can be chosen to assemble the cDNA inserts so that they are in the same translation reading frame. Arnaout et al., J. Clin. Invest. 85:977 (1990). A suitable basic expression vector can be used as a vehicle for the 3,048 bp complete cDNA fragment encoding the human CD11b peptide; the recombinant cDNA can be expressed by transection into, e.g., COS-1 cells, according to conventional techniques, e.g., the techniques generally described by Aruffo et al., Proc. Nat'l. Acad. Sci. USA 84:8573 (1987) or expressed in E. coli using standard techniques. Smith et al., Gene 67:31 (1988).

Isolation of CD11b Peptide from Mammalian Cells

The CD11b protein can be purified from the lysate of transfected COS-1 cells, using affinity chromatography and lentil-lectin Sepharose and available anti-CD11b monoclonal antibody as described by Pierce et al. (1986) supra and Arnaout et al., Meth. Enzymol. 150:602 (1987).

If the desired CD11b peptide is shorter than the entire protein, DNA encoding the desired peptide can be expressed in the same mammalian expression vector described above using the selected DNA fragment and the appropriate restriction enzyme site, as outlined above. The selected DNA fragment may be isolated according to conventional techniques from one of the CD11b cDNA clones or may be synthesized by standard polymerase chain reaction amplification, as described above. See also Saiki et al., (Science 239:487, 1988).

Characterization of the CD11b Polypeptide

The coding sequence of the complete CD11b protein is preceded by a single translation initiation methionine. The translation product of the single open reading frame begins with a 16-amino acid hydrophobic peptide representing a leader sequence, followed by the NH₂-terminal phenylalanine residue. The translation product also contained all eight tryptic peptides isolated from the purified antigen, the amino-terminal peptide, and an amino acid hydrophobic domain representing a potential transmembrane region, and a short 19-amino acid carboxy-terminal cytoplasmic domain (FIG. 1 illustrates the amino acid sequence of CD11b; SEQ ID NO: 43). The coding region of the 155-165 kD CD11b (1,136 amino acids) is eight amino acids shorter than the 130-150 kD alpha subunit of CD11c/CD18 (1,144 amino acids). The cytoplasmic region of CD11b contains one serine residue that could serve as a potential phosphorylation site. The cytoplasmic region is also relatively rich in acidic residues and in proline (FIG. 1). Since CD11b/CD18 is involved in the process of phagocytosis and is also targeted to intracellular storage pools, these residues are candidates for mediating these functions. The long extracytoplasmic amino-terminal region contains three or four metal-binding domains (outlined by broken lines in FIG. 1) that are similar to Ca²⁺-binding sites found in other integrins. Each metal binding site may be composed of two noncontiguous peptide segments and may be found in the four internal tandem repeats formed by amino acid residues 358-412, 426-483, 487-553, and 554-614. The portion of the extracytoplasmic domain between Tyr⁴⁶⁵ and Val⁴⁹² is homologous to the fibronectin-like collagen binding domain and IL-2-receptor. The extracytoplasmic region also contains an additional unique 187-200 amino acid domain, the A domain, between Cys¹²⁸ to Glu³²¹, which is not present in the homologous (a) subunits of fibronectin, vitronectin, or platelet IIb/IIIa receptors. This sequence is present in the highly homologous CD11c protein (α of p150,95) with 64% of the amino acids identical and 34% representing conserved substitutions. Arnaout et al., J. Cell Biol. 106:2153, 1988; Arnaout et al. Blood 75:1037 (1990). It is known that both CD11b/CD18 and CD11c/CD18 have a binding site for complement fragment C3 and this unique region may be involved in C3 binding. This region of CD11b also has significant homology (17.1% identity and 52.9% conserved substitutions) to the collagen/heparin/platelet GpI binding regions of the mature von Willebrand factor (domains A1-A3). The A domain is also homologous to a region in CD11a. Larson et al., J. Cell Biol. 108:703 (1989). The A domain is also referred to as the L domain or the I domain. Larson et al., supra (1988); Corbi et al., J. Biol. Chem. 263:12,403 (1988).

CD11b Peptides

The following peptides can be used to inhibit CD11b/CD18 activity: a) peptides identical to the above-described A domain of CD11b, or a portion thereof, e.g., DIAFLIDGS (SEQ ID NO:32), FRRMKEFVS (SEQ ID NO:33), FKILVVITDGE (SEQ ID NO:34), DGEKFGDPLGYEDVIPEADR (SEQ ID NO:17), or VIRYVIGVGDA SEQ ID NO:35); b) peptides identical to the above-described fibronectin-like collagen binding domain, or a portion thereof, e.g., YYEQTRGGQVSVCPLPRGRARWQCDAV (SEQ ID NO:38); c) peptides identical to one or more of the four metal binding regions of CD11b, or a portion thereof, e.g., DVDSNGSTD (SEQ ID NO:46), DVNGDKLTD (SEQ ID NO:47), DLTMDGLVD (SEQ ID NO:48), DSDMNDAYL (SEQ ID NO:49); d) peptides substantially identical to the complete CD11b; or e) other CD11b domains, e.g. KSTRDRLR (SEQ ID NO:15).

Also of interest is a recombinant peptide which includes part of the A domain, e.g, NAFKILVVITDGEKFGDPLGYEDVIPEADREGV (SEQ ID NO: 50). The A domain binds iC3b, gelatin, and fibrinogen and binding is disrupted by EDTA. The A domain also binds both Ca²⁺ and Mg²⁺. This result unexpected since the A domain lies outside of the region of CD11b previously predicted (Arnaout et al., J. Cell Biol. 106:2153, 1988; Corbi et al., J. Biol. Chem. 25:12403, 1988) to contain metal binding sites.

Protein Sequences

Kishimoto et al., Cell 48:681 (1987) disclose the nucleotide sequence of human CD18. Arnaout et al., J. Cell Biol. 106:2153 (1988); Corbi et al., J. Biol. Chem. 263:12403 (1988); and Hickstein et al., Proc. Nat'l. Acad. Sci. USA 86:275 (1989) disclose the nucleotide sequence of human CD11b. Larson et al., J. Cell. Biol. 108:703 (1989) disclose the nucleotide sequence of CD11a. Corbi et al., EMBO J. 6:4023 (1987) disclose the nucleotide sequence of CD11c. Moyle et al., J. Biol. Chem. 269:10008, 1994 discloses the sequence of Ancylostoma caninum neutrophil adhesion inhibitor). The sequences of the various β subunits are provided by the following references: β1 (Argraves, W. S. et al., (1989) Cell 58, 623-629); β2 (Kishimoto, T. K. et al., (1987) Cell 48, 681-690); β3 (Fitzgerald, L. A. et al., (1987) J. Biol. Chem. 262, 3936-3939); β4 (Suzuki, S. et al., (1990) EMBO J. 9, 757-763); β5 (McLean, J. W. et al., (1990) J. Biol. Chem. 265, 17126-17131); β6 (Sheppard, D. et al., (1990) J. Biol. Chem. 265, 11502-11507); β7 (Yuan Q. et al., (1990) Int. Immuno. 2, 1097-1108); β8 (Moyle et al., (1991) J. Biol. Chem. 266, 19650).

Identification of Antagonists

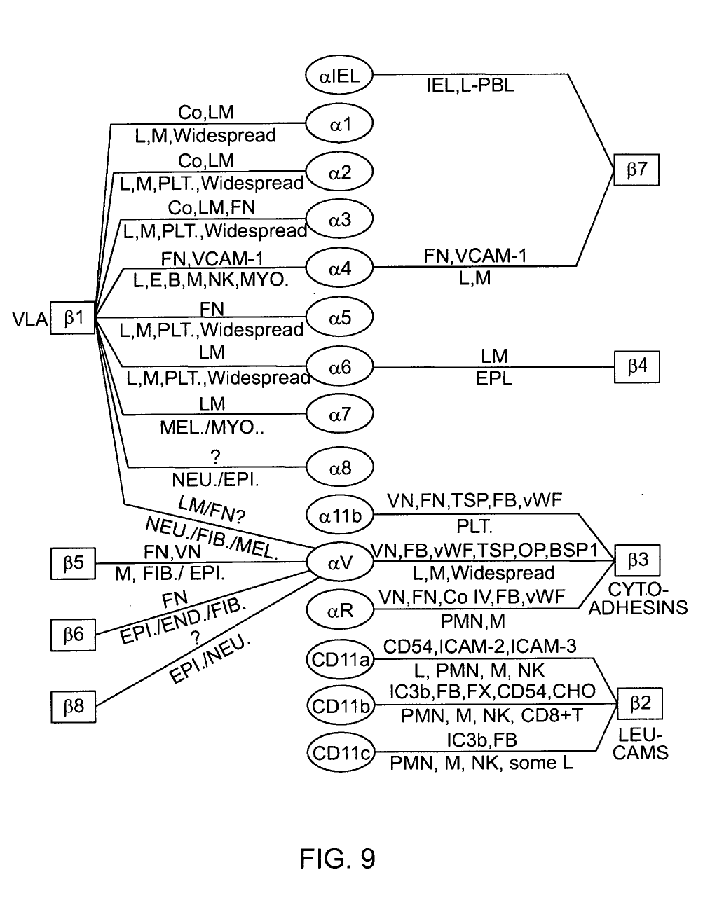

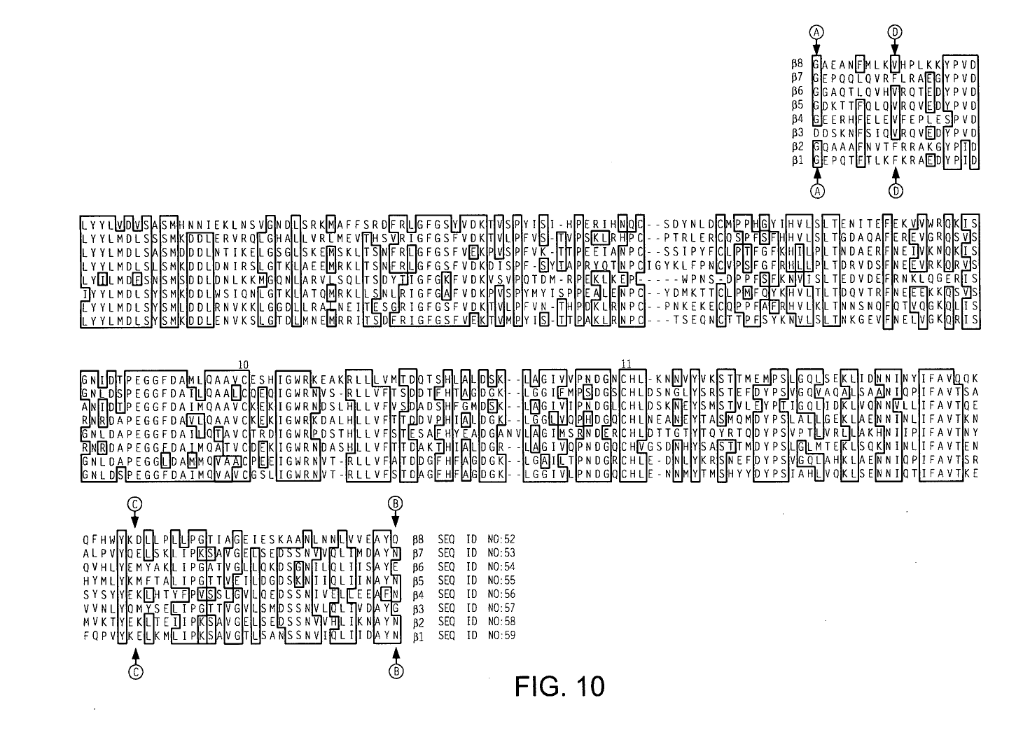

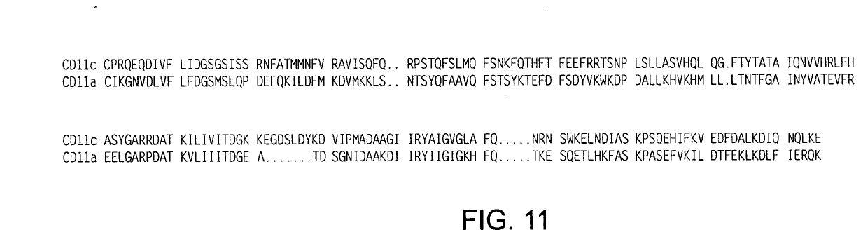

The screening methods of the invention employ an intact integrin A-domain or a ligand-binding fragment thereof. The A-domain of CD11b is described above. The A-domains CD11a and CD11c are depicted in FIG. 11. The A-domains of integrin β subunits β1, β2, β3, β4, β5, β6, β7, and β8 are presented in FIG. 10. These A-domains, or ligand binding fragments thereof, can be used in the methods of the invention to identify antagonists of immunological reactions mediated by their corresponding integrin. Thus, CD11b and β2 A-domain (or ligand-binding fragments thereof) are useful for identifying antagonists of CD11b/CD18 mediated reactions. In assays requiring the use of a ligand which binds the integrin, the preferred ligand is a ligand which is a naturally-occurring ligand of the integrin. A naturally-occurring ligand of an integrin is a ligand which interacts with the integrin as part of an cell-cell, cell-matrix, or matrix-matrix interaction. FIG. 9 is a schematic illustration of the subunit composition of a number of integrins. Also shown in FIG. 9 are some of the ligands which naturally bind each integrin.

The experiments described are specific examples of the identification of antagonists of cell-cell, cell-matrix, or matrix-matrix interactions mediated by integrins which include an A-domain using the methods of the invention. In first series of experiments demonstrate that an antagonist of CD11b/CD18, Ancylostoma caninum neutrophil adhesion inhibitor (NIF) can be identified using a screening method employing the CD11b A-domain. In the second series of experiments screening methods of the invention are used to identifying a ligand-binding fragment of CD11b A-domain which antagonizes binding of complement iC3b to CD11b/CD18. These examples are meant to illustrate, not limit, the invention.

The screening methods of the invention can be used to quickly screen libraries of peptides, antibodies, or small molecules to identify antagonists.

Also described are a number of assays which can be used to further characterize antagonists identified by the methods of the invention.

Binding of NIF to the CD11b A-domain

For this experiment recombinant CD11b A domain (rCD11bA) and recombinant CD11a A domain (r11aA) were expressed as GST fusion proteins as described below, and used as such or after thrombin cleavage.

The recombinant peptides were immobilized and the binding of biotinylated recombinant NIF was measured. Biotinylated rNIF bound directly and specifically to immobilized r11bA. Binding of rNIF to this domain was characterized by a rapid on rate, and a slow off rate, that were almost identical to those characterizing NIF binding to whole neutrophils (see below). NIF binding to immobilized rA-domain was specific and saturable. Scatchard analysis of this binding yielded an apparent K_{d} of −1 nM, similar to that obtained when the neutrophil-bound native CD11b]CD18 was used (see below). In western blots, biotinylated NIF bound directly to r11bA but not to r11aA, and binding to r11bA was inhibited completely by the mAb 107, and partially by OKM9, but not by 44, 904 or TS1/22, indicating the specificity of r11bA-NIF interactions.

The following experiments demonstrate that binding of NIF to recombinant CD11b A-domain is metal dependent. Binding to rCD11b A-domain was measured as described below Binding of NIF to immobilized r11bA required divalent cations, as it was blocked in the presence of EDTA. EDTA was also able to completely reverse r11bA-NIF interaction even when added one hour after the complex is formed. NIF bound to r11bA in VBSG⁻⁻ buffer under these conditions, and binding was not significantly affected by Chelex treatment of VBSG⁻⁻ or by addition of Ca²⁺, Mg²⁺, or Mn²⁺ each at 1 mm. Addition of EGTA at 1 mM to the VBSG⁻⁻ buffer reduced NIF binding only marginally, indicating that binding can occur in the absence of Ca²⁺. As the other cations (e.g. Mg²⁺, Mn²⁺) cannot be selectively chelated, we cannot exclude that binding of NIF to r11bA can occur in presence of Ca²⁺ alone. Since binding is abolished by EDTA, trace amounts of other divalent cations (derived from the buffer salts, gelatin, glucose or BSA) are essential. The divalent cations appear to be required at least at the level of the A-domain, since the mutant r11bA (D140GS/AGA) that lacks the metal binding site(s) did not bind NIF even in the presence of 1 mM each of Ca²⁺ and Mg²⁺. Binding of NIF to the A-domain was not affected by temperature as in whole cells. Fluid-phase r11bA, but not its fusion partner GST, abolished biotinylated rNIF binding to human neutrophils or to immobilized r11bA in a dose dependent manner, with half-maximal inhibition seen at ˜1 nM in each case, reflecting the lack of significant structural differences between the adsorbed and soluble forms of r11bA.

To identify the site in r11bA involved in NIF binding, eleven overlapping peptides spanning the A-domain were synthesized and tested for their ability to inhibit NIF binding to immobilized r11bA. We found that the two contiguous peptides (A6 and A7) inhibited binding of rNIF to rCD11b A-domain dramatically. A scrambled form of A7 had no such effect. Two additional peptides (A1 and A12), located at the beginning and end of the domain had moderate and weak inhibitory effects respectively. Dose response curves revealed that while combining A6 and A7 each at 161 mg/ml (80-115 mM) achieved complete inhibition of biotinylated NIF binding to r11bA, addition of A1 (but not A12) produced a shift in the binding curve to the left suggesting that A1 within the recombinant A-domain also contribute to NIF-r11bA interaction. Some peptides (A7, A7M, A3, A11) adsorbed well to microtiter plates, allowing an assessment of the direct binding of rNIF to these peptides. Biotinylated rNIF bound to immobilized A7 peptide but not to A3 and A11. Binding of NIF to A7 was not affected when the aspartate residue at position 242 (involved in metal coordination in r11bA and CD11b/CD18) is replaced with alanine. Direct binding of rNIF to A6, A1, A12 could not be tested because these peptides did not absorb to plastic wells.

Generation and purification of CD11 A-domain recombinant proteins: The GST-fusion proteins were produced in Escherichia coli using standard methods (see Machishita et al., Cell 72:859, 1993; Ueda et al., Proc. Nat'l Acad. Sci USA 91:10684, 1994). The GST fusion proteins were purified by affinity chromatography using the method of Smith et al. (Gene 67:31, 1988) and used as fusion proteins or cleaved with thrombin (Gene 67:31, 1988) to release the A-domains. Recombinant purified NIF (rNIF) provided by Drs. Matthew Moyle, and Howard R. Soule (Corvas International Inc., San Diego). A recombinant soluble form of human CD54 (containing all five Ig domains but lacking the intramembranous and cytoplasmic regions) was provided by Dr. Jeffrey Greve (Miles Research Center, West Haven, Conn.; Greve et al, Cell 56:839, 1989). Recombinant protein concentrations were determined using the protein assay kit from BioRad Laboratories (Melville, N.Y.) and analyzed by Coomassie staining after electrophoresis on denaturing polyacrylamide gels (Laemmli, Nature (Lond). 227:680, 1970). Each recombinant protein reacted with several blocking monoclonal antibodies (44, 904, OKM9 and 107 in the case of the r11bA, and TS1/22 and L1 in the case of the r11aA; Ueda et al., Proc. Nat'l Acad. Sci USA 91:10684, 1994), confirming the identity of the polypeptides.

Reagents. Synthetic Peptides, and Antibodies: Restriction and modification enzymes were purchased from New England Biolabs (Beverly, Mass.), Boehringer Mannheim Biochemicals (Indianapolis, Ind.) or BRL (Gaithersburg, Md.). The vector pGEX-2T was obtained from Pharmacia LKB Biotechnology, Inc. (Piscataway, N.J.). Murine mAbs directed against human CD11b [44 (Arnaout et al., J. Clin. Invest. 72:171, 1983); 904 (Dana et al., J. Immunol. 137:3529, 1986); OKM9 (Wright et al., Proc. Nat'l Acad. Sci. USA 80:5699, 1983)], CD11a [TS1/22 (Sanchez-Madrid et al., J. Exp. Med. 158:1785, 1983)], CD18 [TS1/18 (Sanchez-Madrid et al., J. Exp. Med. 158:1785, 1983)], and CD11c [L29 (Lanier et al., Eur. J. Immunol. 15:713, 1985)] were prepared as described in the cited references. The mAb 107 was prepared by immunizing BALB/c mice with pure recombinant CD11b A-domain. This mAb reacts with CD11b but not CD11a A-domain by ELISA, immunoprecipitates CD11b/CD18 from neutrophil extracts, and and binds to neutrophils by FACS analysis. Synthetic peptides can be obtained commercially and purified by HPLC according to standard techniques. In some cases selected peptides were subjected to amino acid analysis. Synthetic peptides described herein were soluble in water at 1 mg/ml.

Immobilization of Recombinant Proteins and Polypeptide: Purified rA-domain preparations (1 μg/well), soluble CD54, human fibrinogen (Sigma Chemical Co., St. Loius, Mo.), gelatin (BioRad-Laboratories) or BSA (Calbiochem-Behring Corp.) (each at 10 μg/well) or selected A-domain-derived peptides (10 μg) were added to Immulon-2 96-well microtiter plates (Dynatech Labs, Chantilly, Va.) overnight. Quantitation of adsorbed wild-type and mutant A-domain and synthetic peptides was done using the mAb 44 in an ELISA, and the BCA kit (from Pierce Chemical Co., Rockford, Ill.), respectively. Wells were then washed with phosphate-buffered-saline (PBS), pH 7.4 without metals, and blocked with 1% BSA for one hour, washed again in binding buffer and used immediately in the functional assays.

Biotinylation of recombinant NIP and Measurement of Binding to Immobilized Peptides: Recombinant NIP was labeled with sulfo-NHS-biotin as described by the manufacturer (Pierce Chemical Co.). To measure binding of biotinylated rNIF to immobilized r11bA, increasing concentrations of biotinylated rNIF in VBSG⁺⁺ (veronal-buffered saline, pH 7.4, containing 0.1% gelatin, 1 mM CaCl₂, 1 mM MgCl₂) in the absence or presence of 100-fold unlabeled rNIF, were added to A-domain-coated 96-well microtiter wells, and incubated at RT for 60 minutes. Wells were then washed, incubated with alkaline phosphatase-coupled avidin, washed again, developed with substrate and quantified colormetrically using a microplate reader. To evaluate the ability of anti-CD11b A-domain mAbs to block biotinylated NIP binding to immobilized r11bA, coated wells were preincubated with the mAbs (each at 100 mg/ml or 1:100 dilution of ascites) for 15 minutes at RT. Biotinylated NIF (50 ng/ml final concentration) was then added, and incubation continued for an additional hour. To assess the ability of fluid-phase r11bA or GST to block biotinylated NIP binding to immobilized r11bA, each was preincubated at 7 mg/ml with biotinylated NIP (50 ng/ml final concentration) in a total volume of 50 μl for 15 minutes at RT, followed by incubation of this mixture with the r11bA-coated wells for an additional hour. In experiments where the effects of divalent cations on biotinylated rNIF binding to immobilized rCD11b A-domain were measured, VBSG⁻⁻ buffer (veronal-buffered saline, pH=7.4, containing 0.1% gelatin) containing 1 mM of Ca²⁺, Mg²⁺, Mn²⁺, EDTA, EGTA, EGTA plus 1 mM MgCl₂, or 1 mM MnCl₂. In these experiments, BSA-blocked A-domain containing wells were first washed with buffer containing 10 mM EDTA (to remove protein-bound cations), then washed with the respective binding buffer. The effect of temperature was evaluated in the presence of the standard divalent cation mixture at 37° C., 22° C. and at 4° C. with saturating amounts of biotinylated rNIF (200 ng/ml).

The kinetics of rNIF-neutrophil or rNIF-A-domain interactions were determined as described by Lowenthal et al. (In Current Protocols in Immunology, Colgan et al., eds. Vol. 1:6.0.1-6.1.15, 1992). Neutrophils or immobilized rA-domains were each incubated with half-saturating concentrations of biotinylated rNIF (20 ng/ml and 40 ng/ml for neutrophils and immobilized rA-domain, respectively), in the absence or presence of 100-fold molar excess of unlabeled rNIF at 4° C. (with neutrophils) or at RT (with immobilized rA-domain). The specific binding of biotinylated rNIF was determined at various times as described above, and plotted vs. time. The time required to reach equilibrium was one hour. The value for t_{1/2} of association was determined graphically from the association plot. To determine dissociation rates, neutrophils or immobilized A-domains were incubated for one hour with the respective half-saturating concentrations of biotinylated rNIF mentioned above, in the absence or presence of 100 fold molar excess of unlabeled rNIF, at 4° C. (for neutrophils) or at RT (for immobilized A-domain). Afterwards, neutrophils were washed twice in VBSG⁺⁺ and incubated in 4 ml of this buffer on ice with shaking. At various time points, aliquots were removed, centrifuged and the amount of specifically bound rNIF measured. For immobilized r11bA-domain, wells were washed twice and incubated with 300 μl of VBSG⁺⁺ per well at RT with shaking. At various time points, buffer was removed and specific binding was measured. The dissociation rates in each case were determined by plotting −ln(B/B_{eq}) versus time, where B and B_{eq} represent respectively the fraction of rNIF bound to cells (or to immobilized r11bA-domain) at time t, and at equilibrium. The value for t_{1/2} of dissociation was calculated according to the formula t_{1/2}=ln2/K_{off} (Lowenthal et al., In Current Protocols in Immunology, Colgan et al., eds. Vol. 1:6.0.1-6.1.15, 1992).

Characterization of the Effect of NIF Integrin Function

Having identified NIF as a protein which can bind to CD11b A-domain, a series of additional assays can be employed the characterize the effect of NIF on integrin function. These characterization assays, described in more detail below, can be used to assess any-CD11b A-domain binding molecule identified using the method of the invention.

Binding of NIF to Neutrophils: The time course of association of biotinylated NIF with neutrophils at 4° C. (to avoid endocytosis) was performed as described below. These measurements revealed a rapid uptake, with maximum levels achieved within 60 minutes, and with a t_{1/2} at 15 minutes, and was completely inhibited in the presence of 100-fold molar excess of unlabeled NIF at each time point.

Upon washing and dilution of cells preincubated for one hour at 4° C. with biotinylated rNIF, the cell-associated rNIF slowly dissociated with a t_{1/2} of ˜7.6 hours. Thus, the association of rNIF with neutrophils is reversible and characterized by rapid binding and very slow dissociation. The slow dissociation rate permitted the use of biotinylated rNIF under the conditions described to evaluate its interaction with whole cells and with protein fragments. Incubation of increasing concentrations of biotinylated rNIF with resting or activated neutrophils at 4° C., revealed a predominantly saturable component, with the non-saturable (non-specific) fraction (obtained in the presence of 100-fold molar excess of unlabeled rNIF) accounting for less than 10% of the total binding. A Scatchard plot of the binding data indicated a linear relationship in both resting and activated cells. Both cell types bound NIF with approximately similar affinities (apparent dissociation constants K_{d}, ranging from 0.35 to 1.3 nM), suggesting that the 12-fold increase in NIF binding to activated vs resting cells is primarily due to an increase in the number of NIF binding sites induced by cell activation.

Biotinylation of Recombinant NIF and Measurement of Binding to Neutrophils

100 μg of rNIF were labeled with sulfo-NHS-Biotin as described above. rNIF binding to resting or stimulated human neutrophils (pretreated with 10⁻⁶ M f-met-leu-phe, for 15 minutes at 37° C., then washed) was measured. Increasing amounts of biotinylated rNIF in the absence or presence of 100-fold molar excess of unlabeled rNIF were incubated on ice for one hour with 1×10⁶ neutrophils in VBSG⁺⁺ in a total volume of 50 ml. Cells were then washed and incubated with phycoerythrin-coupled avidin (Sigma Chemical Co.) under similar conditions, washed again, fixed in 1% paraformaldehyde in PBS, and analyzed using FACScan (Becton Dickinson Co., Mountain View, Calif.). Mean channel fluorescence for each sample was then expressed as a function of the amount of biotinylated rNIF used. Background binding of phycoerythrin-streptavidin alone to neutrophils was subtracted (2.8 fluorescent units). Specific binding was obtained by subtracting total binding from that seen in the presence of excess unlabeled rNIF, and the values plotted according to Scatchard (Ann. N.Y. Acad. Sci. 51:660, 1949). To determine the effect of unlabeled fluid-phase-r11bA or GST on rNIF binding to neutrophils, each was preincubated at varying concentrations with biotinylated rNIF (20 ng/ml, final concentration) for 15 minutes on ice before addition of the mixture to neutrophils. The effect of mAbs on biotinylated NIF binding to neutrophils was assessed by preincubating the neutrophils with 100 μg/ml of each mAb at 4° C. for 15 minutes before addition of biotinylated NIF (20 ng/ml). The incubation then continued for one hour, followed by processing of cells for FACS analysis as described below.

Effects of rNIF on Neutrophil Ligand Binding and Phagocytosis

The effects of rNIF on CD11b/CD18-mediated neutrophil binding to the physiologic ligands complement iC3b, fibrinogen, and CD54 were measured. rNIF inhibited binding of EAiC3b to recombinant human CD11b/CD18 (expressed in COS cells) in a dose-dependent manner with complete inhibition achieved at 3 mg/ml (IC₅₀ of −5 nM). rNIF also abolished iC3b-dependent phagocytosis of serum-opsonized oil red O particles by human neutrophils.

Binding of f-met-leu-phe-activated fluoresceinated neutrophils to microtiter wells coated with human fibrinogen or soluble CD54 was also inhibited significantly in the presence of NIF (5 μg/ml). Inhibition of neutrophil binding to fibrinogen was incomplete even at high NIF concentrations (50 mg/ml). CD54 binds to both CD11a/CD18 and CD11b/CD18. Complete inhibition of neutrophil-CD54 interactions therefore requires the simultaneous use of mAbs directed against both antigens. Although NIF did not inhibit neutrophil binding to CD54 when used alone, it abolished this binding when combined with an anti-CD11a mAb.

Preparation of complement C3-coated erythrocytes: Sheep erythrocytes were incubated with 1:240 dilution of rabbit anti-sheep erythrocyte antiserum (Diamedix Corp., Miami, Fla.) for 30 min at 37° C. to generate IgM-coated sheep erythrocytes (EA). EAiC3b was prepared using C5-deficient human serum (Sigma Chemical Co., St. Louis, Mo.) at 1:10 dilution (60 min at 37° C.). EAiC3b cells were washed and stored in isotonic VBSG⁺⁺ to which Soybean Trypsin Inhibitor (STI; Worthington Biochemical Co., Freeton, N.J.) was added at 1 mg/ml. EAiC3b (at 1.5×10⁸ cells/ml) were labeled with 5-(and-6)-carboxy fluorescein (Molecular Probes, Eugene, Oreg.) at 1:100 dilution of a 10 mg/ml stock for 5 min on ice and washed before use in the binding studies.

Recombinant CD11b/CD18 binding to EAiC3b: Binding of EAiC3b to recombinant, membrane-bound CD11b/CD18 expressed on COS cells was performed as described by Machishita et al. (Cell 72:857, 1993). To assess the effect of NIF on this interaction, EAiC3b binding was performed in the absence and presence of increasing amounts of NIF. After incubation, cells were washed, examined briefly by light microscopy then solubilized with 1% SDS-0.2 N NaOH. Fluorescence was quantified (excitation wavelength, 490 nm, emission wavelength, 510 nm) on each sample using a SLM 8000 fluorometer (SLM Instruments, Urbana, Ill.) as described by Machishita et al. (Cell 72:857, 1993).

Neutrophil binding to fibrinogen and CD54: Human neutrophils were purified as described by Boyum et al. (Scand. J. Clin Lab. Invest. 97 (Suppl.):77, 1968). Binding of neutrophils to CD54-coated or fibrinogen-coated. 96-well microtiter plates was performed as follows: Neutrophils (8×10⁶/ml) were labeled with 5-(and-6)-carboxy fluorescein (Molecular Probes, Eugene, Oreg.) at 1:100 dilution of a 10 mg/ml stock for 5 min on ice and washed in M199 medium containing an additional 1 mM MgCl₂, 1 mM CaCl₂ and 0.1% BSA (MB) before use. Fluoresceinated neutrophils (25 μl of 8×10⁶/ml) were added to each well containing 25 μl of buffer alone or containing 2×10⁻⁶ M f-met-leu-phe. The plates were centrifuged at RT (800 rpm in a Sorvall RT 6000B) for 30 s, and incubated for only five min at RT, to avoid cell spreading, a fact confirmed by visual inspection of the cells at the end of this incubation period. Wells were washed three times with 100 ml of MB each, examined by light microscopy, then solubilized with 1% SDS/0.2N NaOH and fluorescence quantified. To evaluate the effects of mabs and NIF on binding, mAbs (each used at 1:100 dilution of ascites) or NIF (used at 5 mg/ml final concentration) were preincubated with fluoresceinated neutrophils for 15 minutes at 4° C. prior to the binding reaction.

Phagocytosis Assays: Phagocytosis of serum opsonized oil red O (ORO) particles was performed essentially as described by Arnaout et al. (N. Engl. J. Med. 306:693, 1982). To determine the effect of rNIF or the anti-CD11b mAb 44 on phagocytosis, rNIF (at 4 μg/ml) or 44 (at 10 μg/ml) were preincubated with neutrophils for 10 minutes at RT prior to addition of opsonized ORO. The reactants were prewarmed for 2 minutes at 37° C. before mixing. Incubation was then commenced for 5 min at 37° C. with continuous shaking in a water bath. The reaction was stopped by addition of 1 ml of ice-cold PBS containing 1 mM. N-ethyl-maelamide (NEM), followed by two washes. The cell pellet was examined visually for its red color (reflecting ingestion of the red oil droplets), then solubilized with 0.5 ml of dioxane, and the amount of ORO in the extract quantified by measuring absorption at 525 nm and converted to milligrams of ORO ingested/10⁵ cells/minute. Specific uptake of ORO was determined by subtracting the background (uptake in the presence of 1 mM NEM).

Binding of NIF to CD11b/CD18

Western blots of heterodimeric CD11b/CD18 immunoprecipitated from unlabeled-neutrophils were probed with biotinylated rNIF, and the pattern was compared with biotinylated CD11b/CD18 (generated by surface biotinylation of neutrophils). This analysis showed that rNIF binds to the CD11b but not the CD18 subunit of the CD11b/CD18 heterodimer. rNIF did not bind to the other two b2 integrins CD11a or CD11c expressed on neutrophils.

To determine if CD11b/CD18 is the only receptor on the neutrophil surface that binds to NIF, several anti-CD11b mAbs known to inhibit CD11b/CD18 functions were evaluated for their ability to block the binding of biotinylated NIF to neutrophils. These studies demonstrated that mAb 107 inhibited NIF binding to neutrophils completely. Two other anti-CD11b mAbs, 44 and 904, and the anti-CD11a mAb (TS1/22) had no inhibitory effect.

Surface biotinylation immunoprecipitation and Western blotting: Surface biotinylation of purified human neutrophils was performed on ice by incubating the cells (3×10⁷/ml in PBS) with 0.1 mg/ml final concentration of Sulfo-NHS-Biotin (Pierce Chemcial Co.) for 30 min at 4° C. Afterwards, cells were washed twice in PBS, quenched for 15 min in RPMI on ice and washed once again in PBS. The NP-40-soluble fraction from unlabeled or biotin-labeled cells was used to immunoprecipitate β2 integrins proteins with the anti-CD11a, b, c-specific mAbs (TS1/22, 44, L29, respectively). Immunoprecipitates were electrophoresed on gradient 4-16% polyacrylamide gels in Laemmli buffer, electroblotted onto Immobilon-P membranes and blocked with BSA. Membranes containing immunoprecipitates from surface-biotinylated cells were then probed with HRP-coupled avidin (Sigma Chemical Co.), while those with immunoprecipitates from unlabeled cells were first probed with biotinylated rNIF (at 1 mg/ml), washed then re-probed with HRP-coupled avidin (Sigma Chemcial Co.). Membranes were developed using the ECL system from Amersham Corp. (Arlington Heights, Ill.).

NIF as a Disintegrin

Taken together the above-described experiments demonstrate that hookworm-derived NIF is a specific CD11b/CD18 antagonist that binds to neutrophils through the CD11b A-domain and inhibits their ability to recognize several CD11b/CD18 ligands and to mediate phagocytosis. The binding of NIF to the CD11b A-domain is selective, of high affinity and divalent cation-dependent. The NIF binding site in r11bA partially overlaps that of human iC3b, the major complement C3 opsonin.

Evidence supporting that CD11b/CD18 is the sole receptor on the neutrophil surface for NIF is based on four types of experiments. First, binding of biotinylated NIF to intact cells was completely blocked by an anti-CD11b/CD18 mAb. Second, probing western blots of detergent extracts from normal or β2 integrin-deficient neutrophils with biotinylated NIF revealed a single specific band, that of CD11b, in normal cell lysates, that was lacking in the genetically-deficient cells. Third, of the three β2 integrins immunoprecipitated from normal neutrophils, only the CD11b subunit reacted with biotinylated NIP in western blots. NIF bound to neutrophil CD11b/CD18 with high affinity (nM range) and inhibited the binding of neutrophils to the CD11b/CD18 ligands iC3b, fibrinogen and CD54. Fourth, soluble r11bA completely blocked the binding of biotinylated NIF to neutrophils. These findings indicate that NIF is a highly selective CD11b/CD18 antagonist.

Previous studies have identified several naturally-occurring proteins, so-called disintegrins, that bind to other integrins with high affinity and block integrin-mediated adhesion (reviewed in Philips et al., Cell 65:359, 1991). Disintegrins isolated from leeches and snake venoms inhibit adhesion-dependent functions such as platelet aggregation when present in low nanomolar concentrations. The majority of disintegrins contain the tripeptide Arg-Gly-Asp and have so far been shown to bind to integrins lacking the A-domain (e.g., members of the β1, β3 and β5 integrin families). Disintegrins interact with their respective receptors through a disintegrin domain, a ˜60 amino acid motif with a characteristic cysteine-rich profile. NIF neither contains an Arg-Gly-Asp sequence, nor the disintegrin motif (Moyle et al., J. Biol. Chem. 269:10008, 1994). The unique structure of NIP probably reflects different structural requirements for antagonists targeting the A-domain-containing integrins. It is interesting to note that the physiologic ligands of CD11b/CD18 such as iC3b, fibrinogen and CD54 do not contain or do not require an Arg-Gly-Asp sequence. NIF may similarly contain a novel motif with cellular counterparts functioning perhaps in regulating important physiologic interactions. Identification of the active site in NIF involved in integrin binding should be very useful in this regard.

The binding site of NIF in CD11b/CD18 is the A-domain. This conclusion is based on the following observations. First, NIF bound to r11bA directly, specifically and with kinetics and affinity very similar to that in whole neutrophils. Second, binding of NIF to immobilized r11bA was blocked by the anti-CD11b A-domain mAb 107 or with excess unlabeled fluid-phase r11bA. Third, fluid-phase r11bA completely blocked the binding of biotinylated NIF to intact neutrophils.

Treatment of Hookworm Disease

By producing a factor, NIF, that blocks CD11b/CD18-mediated functions in neutrophils, hookworms may be able to prevent neutrophil extravasation into infected regions and the destruction of the parasites through their phagocytic and killing abilities. Because rCD11bA inhibits NIF binding to leukocytes in the low nM range whereas its inhibition of iC3b binding to the same cells requires micromolar concentrations, rCD11bA may be useful as such or in a modified form for the treatment of hookworm infection, without producing generalized immunosuppression.

Methods for Identifying Ligand Binding Portions of an Integrin A-domain

The experiments described below illustrate one systematic means for identifying a ligand binding fragment of an A-domain peptide. In this method a series of overlapping peptides spanning the A-domain are created. These peptides are then test for their ability to bind to a selected integrin ligand (preferably a naturally-occurring ligand, e.g., complement iC3b). Both direct and indirect assays are illustrated below. In the direct assay binding of the A-domain peptide fragment to the selected ligand is measured and used as a gauge of the ligand binding ability of the peptide fragment. In the indirect assay the ability of the fragment to inhibit binding of full-length A-domain peptide to a ligand to the full-length A-domain peptide is measured and used as a gauge of the ligand binding ability of the peptide fragment.

Materials: To generate the CD11a A-domain, the respective cDNA was cloned by PCR using CD11a cDNA based oligonucleotides as described by Larson et al. (J. Cell. Biol. 108:703, 1989), inserted in-frame into the BamHI-SmaI restricted pGEX-2T vector (Pharmacia), and the ligated product purified and used to transform. E. coli JM109. Individual bacterial clones containing the cloned cDNA fragment were identified by restriction analysis, and the recombinant protein expressed as a glutathione-S-transferase (GST) fusion protein, purified and released by thrombin (Michishita et al. Cell 72:857, 1993; Smith et al., Gene 67:31, 1988), and analyzed on denaturing 12% polyacrylamide gels. Synthetic peptides were obtained commercially, purified on HPLC, and selective ones were subjected to amino acd analysis.

Erythrocytes (E) coated with rabbit anti-E IgM (EA) or C3b (EAC3b) were prepared as described by Dana et al. (J. Immunol. 73:153, 1984). EAiC3b (erythrocytes coated with iC3b) were generated by treating EAC3b with purified human factors H and I, or alternatively prepared from EA using C5-deficient human serum (Sigma Chemical Co., St. Louis, Mo.). EAiC3b cells were washed and stored in isotonic veronal-buffered saline (VBS²⁺), pH 7.4, containing 0.15 mM calcium-1 mM magnesium (MgCl₂+CaCl₂) and 1 mg/ml Soybean Trypsin Inhibitor (STI; Worthington Biochemical Co., Freehold, N.J.) at 1.5×10⁸ cells/ml. EA, EAC3b or EAiC3b were labeled with 5-(and-6)-carboxy fluorescein (Molecular Probes, Eugene, Oreg.) as described by Michishita et al. (Cell 72:857, 1993).

Immobilization of recombinant proteins and peptides: Purified recombinant A-domain was added to Immulon-2 96-well microtiter plates (Dynatech) overnight. Wells were then washed once with phosphate-buffered-saline, pH 7.4 without metals, and blocked with 1% BSA at room temperature (RT) for one hour, followed by two washings with buffer A (composed of 60% GVBS:VBS²⁺ mixed in a 1:3 ratio; Arnaout et al., in Complement Receptor Type 3 at 602-615, Academic Press, FL) containing 1 mM MnCl₂ or MgCl₂+CaCl₂. All the peptides were stocked at 1 mg/ml in water and similarly adsorbed to Immulon-2 96-well plates. Binding of the anti-CD11b mabs to the coated rA-domain was measured by ELISA and read using a plate reader (Molecular Dynamics).

Erythrocyte binding assays: Fluoresceinated EAiC3b, EAC3b or EA were resuspended to 1.5×10⁸/ml in buffer A, and added (30 ml) to wells containing immobilized proteins or peptides in a total volume of 100 ml. The plates were then briefly centrifuged to settle the erythrocytes, and allowed to incubate at 37° C. for 15 minutes in a humidified incubator with 5% CO₂. For the inhibition studies, E were preincubated with each recombinant protein or pure peptide in the presence of 2% BSA for 5 minutes at RT and added to wells coated with immobilized protein or peptide without washing, unless otherwise indicated. At the end of the binding reactions, wells were washed, examined briefly by light microscopy then solubilized with 1% SDS-0.2 N NaOH. Fluorescence was quantified (excitatory wavelength, 490 nm, emission wavelength, 510 nm) using a SLM 8000 fluorometer (SLM Instruments, Urbana, Ill.). In experiments where the effects of individual divalent cations were measured, Ca²⁺ and Mg²⁺ were replaced with metal-free buffers or with buffers containing each cation at 1. MM, unless otherwise indicated. The effect of temperature was evaluated in the presence of 1 mM MnCl₂ at 37° C. and at 4° C.

Purification and adherence of human neutrophils: Neutrophils were purified as described by Boyum et al. (Scand. J. Clin. Lab. Med. 97(suppl.):77, 1968), resuspended in divalent-cation-free Tris-HCl-saline buffer, pH 7.4 at 5×10⁷/ml and kept on ice until used. Neutrophils (2×10⁵ cells/well) were allowed to adhere to 96-well plates in Iscov's Modified Medium for one hour at 37° C., in a humidified incubator with 5% CO₂. The wells were then washed, and 5 ml of fluoresceinated EAiC3b or EA (at 1.5×10⁸/ml) were added in the presence of 3% BSA, in a total volume of 50 ml, followed by 15 min incubation at 37° C. with 5% CO₂. Wells were then washed and fluorescence quantified as described above.

Flow Cytometrv: Fifteen ml of EAiC3b or EA (each at 1.5×10⁸/ml) were incubated with 15 mg of biotinylated A7 or control peptides in 100 ml of buffer A containing 1 mM MnCl₂ at RT for 10 min and washed once. Streptavidin conjugated phycoerythrin (Sigma) was added to the cell suspension at 1 mg/ml and incubated for 15 min at RT. Washed E were then analyzed by a fluorescence activated cell sorter from Becton Dickinson.

The CD11b A-domain contains an iC3b binding site: The ability of fluoresceinated EAiC3b to bind to a water soluble rCD11b A-domain was examined. The recombinant domain reacted with several mAbs known to inhibit the function of CR3 in whole cells (mAbs: 44, OKM9, and 904). The human rA-domain was immobilized onto 96-well microtiter plates, and incubated with fluoresceinated EAiC3b, EAC3b or EA at 37° C. in the presence of divalent cations. After several washes, the number of bound erythrocytes were quantified using a fluorometer. The rA-domain bound to EAiC3b but not to EAC3b or to EA. The percentage of bound EAiC3b increased progressively as a function of the concentration of the rA-domain used to coat the microtiter wells. Optimal binding occurred upon addition of 20 mg of A-domain, and using 30 ml of EAiC3b (at 1.5×10⁸/ml) per well. Under these conditions EAiC3b binding was easily visible by the naked eye, and was displaced by fluid-phase rA-domain, with half-maximal inhibition observed at ˜1 mM. EAiC3b did not bind to glutathione-S-transferase (GST), or to a homologous rA-domain derived from CD11a/CD18. Furthermore, EAiC3b binding to the rCD11b A-domain was blocked by an anti-CD11b mAb that normally blocks EAiC3b binding to cell-bound CD11b/CD18 (CR3). These data establish the specificity of the interaction between the expressed rCD11b A-domain and iC3b.

Binding of EAiC3b to the rA-domain is divalent-cation dependent but temperature independent: Binding of CD11b/CD18 (CR3) to EAiC3b in whole cells is absent at 4° C. and optimal at 37° C. It also requires the presence of the physiologic divalent cations Mg²⁺ and Ca²⁺, or Mn²⁺. The divalent-cation and temperature dependency of EAiC3b binding to rA-domain was therefor measured. Experiment similar to the binding experiments described above demonstrated that divalent cations were essential for binding. One mM MnCl₂, 1 mM MgCl₂ or a combination of 1 mM MgCl₂ and 0.15 mM CaCl₂ supported this interaction. CaCl₂ alone (0.15-1 mM) was ineffective. No specific binding was observed if divalent cations were omitted, or when EDTA was included in the reaction mixture. Similarly, a single point mutation (D242A) that impairs the ability of the rA-domain to bind divalent-cations (Michishita et al., Cell 7:857, 1993), also impaired its interaction with EAiC3b.

In contrast to the cell-bound heterodimeric receptor, binding of EAiC3b to the rA-domain was temperature-independent. These findings suggest that the temperature dependency of cell-bound CR3 may be required for posttranslational modifications occurring in its cytoplasmic tails, changes in receptor conformation, and/or its cell surface distribution.

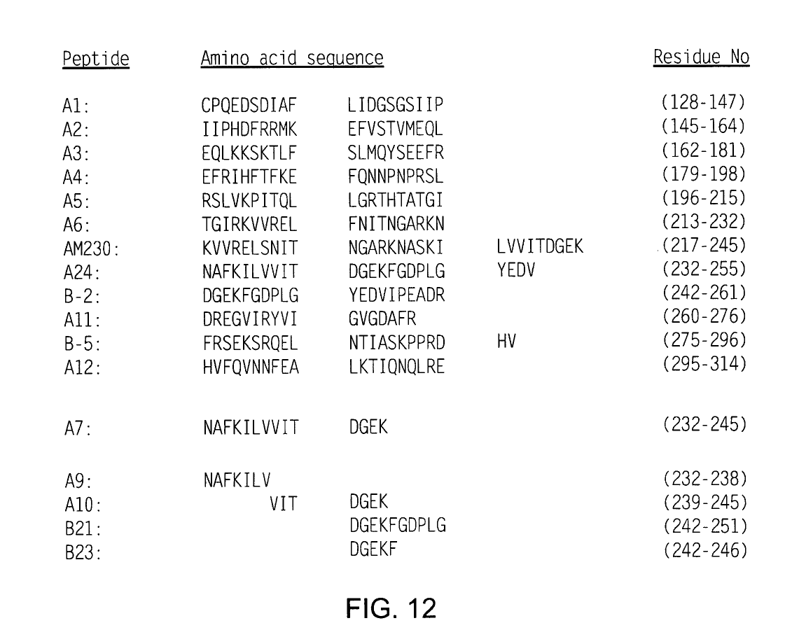

Binding of A-domain-derived peptides to EAiC3b: In order to further define the region within the A-domain that binds EAiC3b, overlapping synthetic peptides spanning the whole A-domain region of CD11b (FIG. 12), were examined for the ability of each to bind directly to EAiC3b and to inhibit EAiC3b binding to the A-domain. Two overlapping peptides, AM230 and A24 (calculated pI of 10.78 and 3.76 respectively) bound directly to EAiC3b but not to EA, and binding was also visible by the naked eye. AM230 and A24 comprised most of the sequence encoded by exon 8 of the CD11b gene, and had a 14 amino acid overlapping region (FIG. 12) when this region (peptide A7) was synthesized on two separate occasions, adsorbed to plastic and tested, it bound EAiC3b directly, specifically and in a dose-dependent manner. No binding was observed when a scrambled form of A7 was used. Fluid-phase biotinylated A7 also bound directly and specifically to EAiC3b, excluding the possibility that the ligand binding observed with the adsorbed peptide is artifactual in nature.

Whereas the interaction of EAiC3b with the rA-domain was divalent-cation dependent, EAiC3b binding to AM230, A24 and A7 was not significantly altered by removal of divalent cations or by inclusion of EDTA. EAiC3b did not bind to wells coated with A7-derived peptides comprising respectively the N-terminal half (A9), the C-terminal half (A10), or the smaller C-terminal peptides B21 and B23. These findings suggest that most of the residues within A7 may be required for iC3b binding. Moreover, microtiter wells precoated with A8, a synthetic peptide from the corresponding A-domain region of CD11a, did not bind to EAiC3b, consistent with the lack of binding of the rCD11a A-domain or of rCD11a/CD18 to EAiC3b.

The lack of direct EAiC3b binding by the other CD11b-derived peptides could be caused by differences in the degree of adsorption of peptides to the plastic wells and/or to lower affinities for iC3b. The ability of the purified peptides to bind EAiC3b indirectly was therefor measured. This was done by determining their effect on binding of EAiC3b to immobilized rA-domain. A7 inhibited binding of EAiC3b to the A-domain in a dose-dependent manner, with half-maximal inhibition at 5 mg/ml (˜3.5 mM). At ≧50 mg/ml (35 mM), A7 inhibited EAiC3b binding to the A-domain completely. This inhibition required the continuous presence of A7, was not secondary to degradation of iC3b or to a toxic effect of the peptide on erythrocytes, since the inhibitory effect was reversible when A7-treated EAiC3b cells were washed prior to their addition to adsorbed rA-domain. The ability of each of the remaining peptides to inhibit EAiC3b-rA-domain interaction was then tested at an approximately three-fold higher peptide concentration (200 mg/ml or 100 mM). At this concentration, none of the other tested peptides (including the CD11a peptide A8 and Sc. A7) significantly inhibited rCD11b A-domain-binding to EAiC3b.

The ability of A7 to inhibit EAiC3b binding to CR3 (CD11b/CD18) expressed by normal human neutrophils was measured under conditions similar to those used in assessing EAiC3b binding to rA-domain. EAiC3b binding to neutrophils is primarily mediated by CR3, but can also occur in vitro through complement receptor type 1 (CR1). The effect of A7 on EAiC3b binding was tested in nearly isotonic conditions and in the presence of blocking concentrations of a polyclonal anti-CR1 antibody (Ross et al., J. Exp. Med. 158:334, 1983). These experiments demonstrated that EAiC3b binding to adherent neutrophils was primarily CR3 mediated under these conditions, since it was inhibited by the anti-CR3 mAb 903, which inhibits iC3b binding selectively. A7 but not the control A-domain-derived peptide A4, significantly inhibited CR3-dependent binding of EAiC3b to neutrophils with 70% inhibition observed at 100 mM and almost complete inhibition seen at 140 mM. These findings indicate that A7 is the major site in CR3 responsible for its interaction with iC3b.

Monoclonal Antibodies

Monoclonal antibodies directed against CD11 or CD18 can be used to antagonize CD11/CD18-mediated immune response. Useful monoclonal antibodies can be generated by using a peptide of the invention as an immunogen. For example, monoclonal antibodies can be raised against the A domain of CD11b, CD11a or CD11c, or the A domain of any of β1-β8.

Anti-CD11b monoclonal antibodies which inhibit iC3b binding (mAb 903), neutrophil adhesive interactions, e.g., aggregation and chemotaxis, (mAb 904), or both activities (mAb44a) have been identified. Other monoclonal antibodies (OKM-1, which inhibits fibrinogen binding, and OKM9) have also been mapped to this region. Dana et al., J. Immunol. 137:3259 (1986). These monoclonal antibodies recognize epitopes in the A domain of CDlb. Dana et al., JASON 1:549 (1990).

Additional useful monoclonal antibodies can be generated by standard techniques. Preferably, human monoclonal antibodies can be produced. Human monoclonal antibodies can be isolated from a combinatorial library produced by the method of Huse et al. (Science, 246:1275, 1988). The library can be generated in vivo by immunizing nude or SCID mice whose immune system has been reconstituted with human peripheral blood lymphocytes or spleen cells or in vitro by immunizing human peripheral blood lymphocytes or spleen cells. The immunogen can be any CD11b or CD18 peptide. Similar techniques are described by Duchosal et al., J. Exp. Med. 92:985 (1990) and Mullinax et al., Proc. Nat'l. Acad. USA 87:8095 (1990).

Peptides derived from the A domain of CD11a, CD11b, or CD11c are preferred immunogens. These peptides can be produced in E. coli transformed by a plasmid encoding all or part of the A domain.

A CD18 peptide can also be used as an immunogen. Three anti-CD18 mabs with anti-inflammatory properties (TS18, 10F12, 60.3) have been identified. Binding each of these antibodies to CD18 can be abrogated by a specific point mutation within a particular region of CD18 (Asp¹²⁸ to Asn³⁶¹ of FIG. 8) (SEQ ID No.: 45). Peptide corresponding to this region can be produced in E. coli using a plasmid encoding the A domain.

Assays for CD11b (or CD11c) Peptides, Heterodimers and Monoclonal Antibodies

CD11b (or CD11c) peptides, heterodimers, and monoclonal antibodies such as those described above, can be tested in vitro for inhibition in one of the following five assays: inhibition of granulocyte of phagocyte adhesion to iC3b-coated erythrocytes or bacteria (iC3b binding), inhibition of phagocytosis, inhibition of monocyte/granulocyte adhesion to endothelium, inhibition of chemotaxis, or inhibition of cell-cell aggregation. These assays can be performed as described in U.S. Ser. No. 08/216,081, hereby incorporated by reference. Alternatively, they may be tested in vivo for controlling damage associated with reduced perfusion or immune injury of tissues, as a result of myocardial infarction, burns, frost bite, glomerulonephritis, asthma, adult respiratory distress syndrome, transplant rejection, onset of diabetes mellitus, ischemia, colitis, shock liver syndrome, and resuscitation from hemorrhagic shock.

Assays for CD11a Peptides, Heterodimers and Monoclonal Antibodies

CD11a peptides, heterodimers and monoclonal antibodies can be tested using the inhibition of endothelial adhesion assay (described above) or a lymphocyte proliferation assay. Arnaout et al., J. Clin. Invest. 74:1291 (1984) describes an assay for inhibition of antigen/mitogen induced lymphocyte proliferation.

In Vivo Model for Testing Peptides and Antagonists

Damage to tissues injured by ischemia-reperfussion (e.g., heart tissue during myocardial infarction) can be minimized by administering to an animal an inhibitor of CD11/CD18 mediated immune response. A peptide of the invention may be tested for in vivo effectiveness using animals, e.g., dogs, which have been induced to undergo myocardial infarction. See, e.g. Simpson et al. supra.

Use

The peptides or monoclonal antibody can be administered intravenously in saline solution generally on the order of mg quantities per 10 kilograms of body weight. The peptide can be administered in combination with other drugs, for example, in combination with, or within six hours to three days after a clot dissolving agent, e.g., tissue plasminogen activator (TPA), Activase, or Streptokinase.

The screening assays of the invention are useful for identifying potential antagonists (inhibitors) of immune reactions mediated by A-domain containing integrins. Accordingly, the screening methods of the invention are highly useful for limiting the number of candidate antagonists which would otherwise have to be subjected to more complicated screening proceedures involving intact integrin heterodimers or animal models.

### Other Embodiments

The invention also feature antagonists identified by the screening assays of the invention.

1-14. (canceled)

15. A method of screening compounds to identify a candidate compound for inhibiting the binding of CD11b/CD18 to a selected ligand of CD11b/CD18, the method comprising:
(a) measuring the binding of a substantially pure polypeptide comprising amino acids 128-320 of SEQ ID NO:43 to the selected ligand in the presence of a test compound;
(b) measuring the binding of the substantially pure polypeptide comprising amino acids 128-320 of SEQ ID NO:43 to the selected ligand in the absence of the test compound;
(c) identifying the compound as a candidate compound for inhibiting the binding of CD11b/CD18 to a selected ligand of CD11b/CD18 if the binding measured in step (a) is less than the binding measured in step (b).

16. The method of claim 15 wherein the substantially pure polypeptide consists essentially of a polypeptide fragment of human CD11b.

17. The method of claim 15 wherein the polypeptide is detectably labeled.

18. A method of screening compounds to identify a candidate compound for inhibiting the binding of CD11b/CD18 to a selected ligand of CD11b/CD18, the method comprising:
(a) measuring the binding of a recombinant polypeptide comprising amino acids 128-320 of SEQ ID NO:43 to the selected ligand in the presence of a test compound;
(b) measuring the binding of the recombinant polypeptide comprising amino acids 128-320 of SEQ ID NO:43 to the selected ligand in the absence of the test compound;
(c) identifying the compound as a candidate compound for inhibiting the binding of CD11b/CD18 to a selected ligand of CD11b/CD18 if the binding measured in step (a) is less than the binding measured in step (b).

19. The method of claim 18 wherein the recombinant polypeptide consists essentially of a polypeptide fragment of human CD11b.

20. The method of claim 18 wherein the recombinant polypeptide is detectably labeled.

21. A method of screening compounds to identify a candidate compound for inhibiting the binding of CD11b/CD18 to a selected ligand of CD11b/CD18, the method comprising:
(a) measuring the binding of a substantially pure polypeptide fragment of human CD11b comprising amino acids 128-320 of SEQ ID NO:43 to the selected ligand in the presence of a test compound;
(b) measuring the binding of the substantially pure polypeptide fragment of human CD11b comprising amino acids 128-320 of SEQ ID NO:43 to the selected ligand in the absence of the test compound;
(c) identifying the compound as a candidate compound for inhibiting the binding of CD11b/CD18 to a selected ligand of CD11b/CD18 if the binding measured in step (a) is less than the binding measured in step (b).

22. The method of claim 21 wherein the substantially pure CD11b polypeptide is detectably labeled.
